(19) United States
(12) Patent Application Publication
(10) Pub. No.: US 2025/0277201 A1
(43) Pub. Date: Sep. 4, 2025

(54) Method for Producing Beta-Trypsin

(71) Applicant: IPSEN BIOPHARM LIMITED, Wrexham (GB)

(72) Inventors: Philip Michael Hunt MARKS, Wrexham (GB); Martin MEWIES, Wrexham (GB); Christopher David WILSON, Wrexham (GB); Alina Anna BUGAJEWSKA-WALLER, Wrexham (GB); David Edwin GRUBER, Wrexham (GB); Gareth LEWIS, Wrexham (GB)

(21) Appl. No.: 18/247,430

(22) PCT Filed: Oct. 1, 2021
(86) PCT No.: PCT/GB2021/052549

§ 371 Date: Mar. 30, 2023

(30) Foreign Application Priority Data
Oct. 1, 2020 (GB) 2015618.8

(51) Int. Cl. C12N 9/76; C07K 1/18; C07K 1/34; C12N 1/20; C12N 9/52; C12R 1/19

(52) U.S. Cl. CPC C12N 9/6427; C07K 1/18; C07K 1/34; C12N 1/205; C12N 9/52; C12R 2001/19; C12Y 304/21004; C12Y 304/24069

(57) ABSTRACT

A method for producing β-trypsin, the method comprising: a) renaturing denatured trypsinogen, thereby producing renatured trypsinogen, wherein the renaturing is carried out in a buffer that comprises L-arginine; b) purifying the renatured trypsinogen by anion exchange chromatography, thereby providing purified renatured trypsinogen; and c) incubating the purified renatured trypsinogen under conditions to promote proteolytic activity of the trypsinogen, wherein trypsinogen is cleaved into β-trypsin by said proteolytic activity; wherein steps a) and b) are carried out under conditions that do not promote the proteolytic activity of trypsinogen; wherein the method does not comprise the addition of a further protease for cleaving trypsinogen into β-trypsin; wherein at least step c) is carried out in a buffer that does not comprise L-arginine; and wherein prior to step c), when present in a buffer that does not comprise L-arginine, the trypsinogen is not subjected to a temperature of >8° C. for more than 38 hours.

### FIELD OF THE INVENTION

The present invention relates to methods for producing recombinant trypsin, a recombinant trypsin composition and uses thereof.

### BACKGROUND

Trypsin is a serine protease found in the digestive system of many vertebrates, where it functions to hydrolyze proteins to break them down into smaller peptides by cleaving peptide chains at the carboxyl side of the amino acids lysine and arginine (except where either is followed by proline). Trypsin originates from the pancreas where it is produced in an inactive (zymogen) form, referred to as trypsinogen. Subsequent to production in the pancreas, trypsinogen enters the small intestine (via the bile duct) where it is converted into active trypsin. Trypsin (previously activated from trypsinogen, for example by enteropeptidase) then cleaves a terminal hexapeptide from trypsinogen to yield a single-chain trypsin protein known as β-trypsin. Subsequent autolysis produces other active forms having two or more peptide chains, such as α-trypsin, which has two peptide chains bound by disulfide bonds. The activated trypsin can then perform its digestive functions.

Trypsin is widely used in biotechnology and research. For example, it is used in the production of insulin in combination with carboxypeptidase B for processing of a proinsulin precursor into insulin. Trypsin is also used during the manufacture of non-cytotoxic clostridial neurotoxins, such as botulinum neurotoxin (BoNT). BoNTs are synthesized as an inactive single-chain polypeptide that is activated post-translationally by a proteolytic cleavage event to form two polypeptide chains joined by a disulphide bond. Cleavage occurs at a specific cleavage site, often referred to as the activation site, that is located between the cysteine residues that provide the inter-chain disulphide bond. Trypsin is commonly used to catalyse this cleavage event and thus, advantageously, it is not necessary to engineer an exogenous cleavage site into a BoNT.

Most commercially available trypsin products are purified from the pancreas of an animal, typically the bovine or pig pancreas. However, purification from such animal sources involves the risk of contamination of the trypsin product with pathogens such as viruses or prions, necessitating excessive purification of the trypsin which can reduce the production yield. Furthermore, animal-derived trypsin is generally not useful for the manufacture of pharmaceutical agents, such as insulin and BoNTs, because such trypsin does not satisfy Good Manufacturing Practice (GMP) requirements.

Therefore, there is a lack of suitable methods for producing trypsin at high yields and of sufficient purity for use in activating proteins to be used within pharmaceutical compositions, or cosmetic compositions.

The present invention solves one or more of the above-mentioned problems.

### SUMMARY OF THE INVENTION

In more detail, the present invention is predicated on the surprising finding that β-trypsin can be produced at high purity and high yield by means of a prokaryotic expression system, in which the trypsinogen zymogen is expressed, and subsequently purified before being activated to provide trypsin. The present inventors have found that use of a prokaryotic expression system (e.g. a prokaryotic host cell) advantageously overcomes problems of production scaling and insufficient protein yield which occurs when using a eukaryotic expression system. For example, expression of trypsin in yeast generally results in low and economically insufficient yields.

By expressing trypsinogen instead of the active form (trypsin), the inventors have found that the protein does not exhibit autolysis during expression and purification, mitigating the issues of self-cleavage and resultant reduction in production yield/purity. Advantageously, where trypsinogen is expressed, the protein forms insoluble inclusion bodies comprising aggregated trypsinogen (not representing the final tertiary structure of the active molecule) which is proteolytically inactive, thus amplifying the ability to avoid autolysis. An insoluble inclusion body is an insoluble (yet stable) aggregate (typically a nuclear or cytoplasmic aggregate) of an expressed protein. The advantages of expressing trypsinogen to form inclusion bodies was highly surprising, as it is generally believed that formation of insoluble inclusion bodies when expressing a protein should be avoided, as the downstream processes required to solubilise and renature protein present in such inclusion bodies was believed to significantly compromise production yields.

Advantageously, the present invention employs a combination of cation exchange chromatography (prior to renaturing trypsinogen) and anion exchange chromatography (subsequent to renaturing trypsinogen). The anion exchange chromatography step is suitably carried out at a pH that is less than the pI of trypsinogen (e.g. such that trypsinogen has a net positive charge). The inventors have surprisingly found that the net charge difference between impurities/contaminants (net negative charge) and trypsinogen (net positive charge) in these conditions allows retention of impurities/contaminants in the anion exchange column and rapid flow through of trypsinogen for collection, leading to an increased removal of host cell derived contaminants (e.g. bacterial endotoxins, host cell protein and host cell DNA) thus a trypsin product of high purity. Indeed, the inventors have demonstrated that a purity level of >90% is readily achievable by methods of the present invention. As such, the present invention aligns with Good Manufacturing Practice (GMP) requirements, and advantageously allows for the production of trypsin which finds utility in activating proteins to be used as part of a pharmaceutical or cosmetic composition. Advantageously, the ‘flow through’ approach applied to the anion exchange chromatography (AEX) step allows for recovery of a high yield (relative to the AEX input) of the target polypeptide (trypsinogen) while contaminants remain bound to the column. Take, by way of example, FIG. 4A “1^{st} confirmation run” that outlines a table of protein yield following various steps of the methods described herein. Notably, the ‘step yield’ (yield relative to input) following AEX/Eshmuno Q is higher than that following any of the other ‘purification steps’ in said “1st confirmation run”, while the ‘total yield’ reduces by just 3%. This observation is indicative of an advantageous ‘positive enrichment’ of trypsinogen due to removal of contaminant.

Furthermore, the present inventors have demonstrated that certain steps, particularly an activation step (e.g. in which trypsinogen is cleaved into β-trypsin by autocleavage), provide improved results in the absence of aggregation inhibitors such as L-arginine. In the case of said activation step, it is believed that the omission of L-arginine allows polypeptides to more readily interact, leading to improved trypsinogen-trypsinogen (or trypsin-trypsinogen) interactions required for activation/cleavage. That being said, the presence of L-arginine in earlier steps (e.g. renaturation) is advantageous. Thus, methods of the invention wherein a step of renaturing trypsinogen is carried out in the presence of L-arginine (where the presence of L-arginine is advantageous to aid re-folding) involve a subsequent step that is carried out in the absence of L-arginine. Such approach involves/necessitates trypsinogen being present in a buffer in which L-arginine is absent for a certain period of time (e.g. during preparation for the activation step), which the inventors have demonstrated can lead to reduced stability of the trypsinogen preparation (see Example 2). Without wishing to be bound by theory, it is believed that in the absence L-arginine, an aggregation inhibitor, the (non-activated) trypsinogen molecules form aggregates and precipitate. Advantageously, however, the inventors have demonstrated that by adhering to certain temperature thresholds and/or maximum ‘hold-times’ (time trypsinogen is held in the absence of L-arginine), such aggregation can be suppressed or even avoided.

Thus, not only have the inventors identified a problem associated with the approach of performing activation absent L-arginine, but they have also demonstrated an advantageous solution to overcome said problem. As such, it remains possible to perform renaturation in the presence of L-arginine (where it is advantageous) yet remove L-arginine prior to the activation step (where it is disadvantageous) without compromising the stability of the trypsinogen preparation.

### DETAILED DESCRIPTION

In a first aspect, there is provided a method for producing β-trypsin, the method comprising:
a) renaturing denatured trypsinogen (or renaturing purified denatured trypsinogen), thereby producing renatured trypsinogen, wherein the renaturing is carried out in a buffer that comprises L-arginine;
b) purifying the renatured trypsinogen by anion exchange chromatography, thereby providing purified renatured trypsinogen; and
c) incubating the purified renatured trypsinogen under conditions to promote proteolytic activity of the trypsinogen, wherein trypsinogen is cleaved into β-trypsin by said proteolytic activity;
wherein steps a) and b) are carried out under conditions that do not promote the proteolytic activity of trypsinogen;
wherein the method does not comprise the addition of a further protease for cleaving trypsinogen into β-trypsin;
wherein at least step c) is carried out in a buffer that does not comprise L-arginine; and
wherein prior to step c), when present in a buffer that does not comprise L-arginine, the trypsinogen is not subjected to a temperature of >8° C. for more than 38 hours.

In a preferable embodiment, the term “wherein prior to step c), when present in a buffer that does not comprise L-arginine, the trypsinogen is not subjected to a temperature of >8° C. for more than 38 hours” means a step subsequent to a step of renaturing denatured trypsinogen (e.g. step a. of said first aspect), optionally including said step of renaturing denatured trypsinogen.

The skilled person will understand that the time for which the polypeptide (trypsinogen) may be subjected to a temperature of >8° C. may vary as a function of temperature. For example, as the temperature increases (e.g. to >10° C., >15° C. etc.) it may be preferable to concomitantly reduce the time that the polypeptide is subjected to such temperature.

In certain embodiments, prior to step c), when present in a buffer that does not comprise L-arginine, the trypsinogen is not subjected to a temperature of:
≥10° C. for more than 30 hours; or
≥15° C. for more than 20 hours; or
≥20° C. for more than 15 hours; or
≥25° C. for more than 5 hours.

In one embodiment, prior to step c), when present in a buffer that does not comprise L-arginine, the trypsinogen is not subjected to a temperature of 15-30° C. for more than 20 hours; preferably the trypsinogen is not subjected to a temperature of 15-30° C. for more than 10 hours; more preferably the trypsinogen is not subjected to a temperature of 15-30° C. for more than 5 hours.

In a yet further preferred embodiment, prior to step c), when present in a buffer that does not comprise L-arginine, the trypsinogen is not subjected to a temperature of 15-30° C. for more than 2 hours.

For example, prior to step c), when present in a buffer that does not comprise L-arginine, the trypsinogen may not be subjected to a temperature of >8° C.

The trypsinogen is preferably one that has been expressed in a prokaryote. In other words, the denatured trypsinogen (e.g. of step a)) preferably originates from a prokaryotic host cell.

One or more of the following steps may be carried out prior to step a) (e.g. of said first aspect):
i. culturing prokaryotic host cells comprising a nucleotide sequence encoding trypsinogen, wherein the nucleotide sequence is operably linked to an inducible promoter;
ii. inducing expression of the trypsinogen by the host cells, thereby forming one or more insoluble inclusion bodies comprising the trypsinogen;
iii. isolating the one or more insoluble inclusion bodies from the host cells;
iv. solubilising the one or more insoluble inclusion bodies, thereby producing denatured trypsinogen; and/or
V. purifying the denatured trypsinogen by cation exchange chromatography, thereby providing purified denatured trypsinogen.

Preferably, prior to step a) (e.g. of said first aspect), denatured trypsinogen may be purified by cation exchange chromatography, thereby providing purified denatured trypsinogen. Said purified denatured trypsinogen may then be renatured during step a) of said method.

The following steps may be carried out prior to step a) (e.g. of said first aspect):
i. culturing prokaryotic host cells comprising a nucleotide sequence encoding trypsinogen, wherein the nucleotide sequence is operably linked to an inducible promoter;
ii. inducing expression of the trypsinogen by the host cells, thereby forming one or more insoluble inclusion bodies comprising the trypsinogen;
iii. isolating the one or more insoluble inclusion bodies from the host cells;
iv. solubilising the one or more insoluble inclusion bodies, thereby producing denatured trypsinogen; and
v. purifying the denatured trypsinogen by cation exchange chromatography, thereby providing purified denatured trypsinogen.

Steps a) and b) (e.g. of said first aspect) may be carried out in the absence of calcium. Additionally or alternatively, steps ii), iii), iv) and/or v) (e.g. of said first aspect, as outlined in the preceding paragraph) may be carried out in the absence of calcium.

In a second aspect, there is provided a method for producing β-trypsin, the method comprising:
a) culturing prokaryotic host cells comprising a nucleotide sequence encoding trypsinogen, wherein the nucleotide sequence is operably linked to an inducible promoter;
b) inducing expression of the trypsinogen by the host cells, thereby forming one or more insoluble inclusion bodies comprising the trypsinogen;
c) isolating the one or more insoluble inclusion bodies from the host cells;
d) solubilising the one or more insoluble inclusion bodies, thereby producing denatured trypsinogen;
e) purifying the denatured trypsinogen by cation exchange chromatography, thereby providing purified denatured trypsinogen;
f) renaturing the purified denatured trypsinogen, thereby producing renatured trypsinogen, optionally wherein the renaturing is carried out in a buffer that comprises L-arginine;
g) purifying the renatured trypsinogen by anion exchange chromatography, thereby providing purified renatured trypsinogen;
h) incubating the purified renatured trypsinogen under conditions to promote proteolytic activity of the trypsinogen, wherein trypsinogen is cleaved into β-trypsin by said proteolytic activity; and
i) isolating the β-trypsin by affinity chromatography
wherein steps f) and g) are carried out under conditions that do not promote the proteolytic activity of the trypsinogen; and wherein the method does not comprise the addition of a further protease for cleaving trypsinogen into β-trypsin;
optionally wherein at least step h) is carried out in a buffer that does not comprise L-arginine;
optionally wherein prior to step h), when present in a buffer that does not comprise L-arginine, the trypsinogen is not subjected to a temperature of >8° C. for more than 38 hours.

Preferably the renaturing (e.g. step f)) is carried out in a buffer that comprises L-arginine.

In a preferable embodiment, the term “wherein prior to step h), when present in a buffer that does not comprise L-arginine, the trypsinogen is not subjected to a temperature of >8° C. for more than 38 hours” means a step subsequent to a step of renaturing denatured trypsinogen (e.g. step f. of said second aspect), optionally including said step of renaturing denatured trypsinogen.

In certain embodiments, prior to step h), when present in a buffer that does not comprise L-arginine, the trypsinogen is not subjected to a temperature of:
≥10° C. for more than 30 hours; or
≥15° C. for more than 20 hours; or
≥20° C. for more than 15 hours; or
≥25° C. for more than 5 hours.

In one embodiment, prior to step h), when present in a buffer that does not comprise L-arginine, the trypsinogen is not subjected to a temperature of 15-30° C. for more than 20 hours; preferably the trypsinogen is not subjected to a temperature of 15-30° C. for more than 10 hours; more preferably the trypsinogen is not subjected to a temperature of 15-30° C. for more than 5 hours.

In a yet further preferred embodiment, prior to step h), when present in a buffer that does not comprise L-arginine, the trypsinogen is not subjected to a temperature of 15-30° C. for more than 2 hours.

For example, prior to step h), when present in a buffer that does not comprise L-arginine, the trypsinogen may not be subjected to a temperature of >8° C.

The term “a buffer that does not comprise L-arginine” means a buffer that comprises substantially no L-arginine. A buffer that comprises substantially no L-arginine may have an L-arginine concentration of less than 50 mM, 25 mM, 10 mM, preferably less than 1 mM. More preferably, the term “a buffer that does not comprise L-arginine” as used herein means a buffer that comprises no L-arginine.

In one embodiment, a step of purifying the renatured trypsinogen by anion exchange chromatography is carried out in a buffer that does not comprise oxidised glutathione (GSSG) and/or reduced glutathione (GSH). For example, a step of purifying the renatured trypsinogen by anion exchange chromatography may be carried out in a buffer that does not comprise GSSG or GSH.

The term “a buffer that does not comprise GSSG” means a buffer that comprises substantially no GSSG. A buffer that comprises substantially no GSSG may have a GSSG concentration of less than 50 mM, 25 mM, 10 mM, preferably less than 1 mM. More preferably, the term “a buffer that does not comprise GSSG” as used herein means a buffer that comprises no GSSG. The term “a buffer that does not comprise GSH” means a buffer that comprises substantially no GSH. A buffer that comprises substantially no GSH may have a GSH concentration of less than 50 mM, 25 mM, 10 mM, preferably less than 1 mM. More preferably, the term “a buffer that does not comprise GSH” as used herein means a buffer that comprises no GSH.

Steps f) and g) (e.g. of said second aspect) may be carried out in the absence of calcium. Additionally or alternatively, steps b), c), d), and/or e) (e.g. of said second aspect) may be carried out in the absence of calcium.

A method of the invention may comprise one or more further purification steps that may or may not be carried out under conditions that promote proteolytic activity of the trypsinogen. However, it is preferred that said further steps are also carried out under conditions that do not promote proteolytic activity of the trypsinogen.

In one embodiment, the step of incubating the purified trypsinogen under conditions to promote proteolytic activity of the trypsinogen (e.g. step c. of the first aspect described above, step h. of the second aspect) is carried out up to 120 mins from providing purified renatured trypsinogen (e.g. up to 120 mins from step b. of the first aspect, step g. of the second aspect); preferably up to 75 mins from providing purified renatured trypsinogen.

In a more preferable embodiment, the step of incubating the purified trypsinogen under conditions to promote proteolytic activity of the trypsinogen is carried out up to 60 mins from providing purified renatured trypsinogen. This may be particularly advantageous where the purified renatured trypsinogen is held at a temperature of 15-25° C. (e.g. 20° C.) prior to the step of incubating the purified renatured trypsinogen under conditions to promote proteolytic activity of the trypsinogen.

It may be particularly advantageous to carry out said step within 120 mins (preferably within 75 mins; more preferably within 60 mins) from providing purified renatured trypsinogen in embodiments where anion exchange chromatography is carried out in the absence of L-arginine (or other stability-enhancing excipient). By working to this timeframe, precipitation/aggregation of trypsinogen (with a concomitant loss of proteolytic activity and self-cleavage into β-trypsin) may be avoided (e.g. which may otherwise occur due to the lack of L-arginine in the purified renatured trypsinogen preparation).

For example, the step of incubating the purified renatured trypsinogen under conditions to promote proteolytic activity of the trypsinogen may be carried out up to 45, 30, or 15 mins from providing purified renatured trypsinogen. Again, this may be particularly advantageous where the purified renatured trypsinogen is held at a temperature of 15-25° C. (e.g. 20° C.) prior to the step of incubating the purified renatured trypsinogen under conditions to promote proteolytic activity of the trypsinogen.

Advantageously, by providing said step of purifying the renatured trypsinogen by anion exchange chromatography (e.g. step g), the inventors have found that a β-trypsin preparation of higher purity and yield may be obtained, when compared with prior art β-trypsin preparations lacking said (additional) chromatography step. This technical effect was totally unexpected, as anion exchange chromatography (usually used for purifying proteins having a low isoelectric point, pI) would not have been expected to provide an advantage during the purification of trypsinogen, which has a high pI of 9.3 (and would thus have a net positive charge or neutral charge at typical pH values used during purification, such that the trypsinogen would not bind to the anion exchange resin). However, the inventors have found that the high pI of trypsinogen may be exploited, as it allows the protein to simply flow-through the anion exchange column, while impurities (e.g. impurities having a net negative charge) are retained and thus separated from the trypsinogen. Thus, the anion exchange chromatography step may be carried out under conditions that provide the renatured trypsinogen with a net positive charge, allowing separation from net negative charge-impurities. As outlined above, the ‘flow through’ approach applied to the anion exchange chromatography (AEX) step allows for recovery of a high yield (relative to the AEX input) of the target polypeptide (trypsinogen) while contaminants remain bound to the column. Take, by way of example, FIG. 4A “1^{st} confirmation run” that outlines a table of protein yield following various steps of the methods described herein. Notably, the ‘step yield’ (yield relative to input) following AEX/Eshmuno Q is higher than that following any of the other ‘purification steps’ in said “1st confirmation run”, while the ‘total yield’ reduces by just 3%. This observation is indicative of an advantageous ‘positive enrichment’ of trypsinogen due to removal of contaminant.

In one embodiment, the anion exchange chromatography step is carried out under conditions in which the renatured trypsinogen has a net positive charge.

A step of purifying the denatured trypsinogen by cation exchange chromatography may (additionally or alternatively) be carried out under conditions in which the renatured trypsinogen has a net positive charge. For example, under such conditions, the trypsinogen binds to the cation exchange resin (which has a negative charge).

Without wishing to be bound by theory, it is believed that by employing this additional purification step prior to the activation step (e.g. step h), the activation step is not impeded by the impurities retained by the anion exchange resin, such that an improved yield of activated β-trypsin may be provided. This is in contrast to prior art methods, where final purification or polishing' steps are employed only post-activation (i.e. the actual trypsin preparation itself is polished).

In a preferable embodiment, the renatured trypsinogen is present in a fraction that does not interact, or only weakly interacts, with the anion exchange chromatography media. For example, the fraction may comprise a pH which is less than the isoelectric point (pI) of the renatured trypsinogen (e.g. the pH may be less than 9.2, preferably ≤9.0, more preferably ≤8.5). A particularly preferred pH is in the range of about pH 6.5-7.5.

The isoelectric point (pI) is a specific property of a given protein. In more detail, the isoelectric point (pI) is defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program. Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use. Throughout the present specification, “pI” means “calculated pI” unless otherwise stated.

For example, the “conditions in which the renatured trypsinogen has a net positive charge” may mean that the anion exchange chromatography step is carried out under a pH of less than 9.2, preferably ≤9.0, more preferably ≤8.5. For example, the pH may suitably be ≥pH 6. 5and ≤8.0, even more suitably ≥pH 6.5 and ≤7.5. For example, renatured trypsinogen (e.g. to be purified by anion exchange chromatography) may be present in a buffer having a pH of less than 9.2, preferably ≤9.0, more preferably ≤8.5.

In preferable embodiment, the pH during anion exchange in a method described herein may be ≥pH 7.3 and ≤7.6.

A method of the invention may comprise a buffer exchange step to provide renatured trypsinogen present in a buffer that lacks a stability-enhancing excipient (such as L-arginine); optionally wherein the buffer exchange step is carried out prior to a step of purifying the trypsinogen by anion exchange chromatography to obtain purified trypsinogen. An example of such buffer (e.g. that lacks a stability-enhancing excipient) is 25-100 mM Tris, preferably 50 mM Tris. The buffer may have a pH of 7.0-8.0, preferably about pH 7.4.

In one embodiment, a step of purifying the renatured trypsinogen by anion exchange chromatography, thereby providing purified renatured trypsinogen (e.g. step g)) is carried out at a temperature of less than 15° C. For example, a temperature of 1-10° C.; more preferably a temperature of 2-8° C. Alternatively, a step of purifying the renatured trypsinogen by anion exchange chromatography, thereby providing purified renatured trypsinogen (e.g. step g)) may be carried out at a temperature of at least 15° C. For example, a temperature of 15-25° C.; more preferably a temperature of 20-25° C.

Advantageously, the volume of the renatured trypsinogen may be reduced prior to anion exchange chromatography, reducing the volume to be subjected to purification.

In one embodiment, the method comprises subjecting the renatured trypsinogen (e.g. prior to anion exchange chromatography) to a volume reduction step, for example thereby providing renatured trypsinogen having reduced volume. Additionally or alternatively, the method may comprise subjecting the purified renatured trypsinogen (e.g. subsequent to anion exchange chromatography) to a volume reduction step, for example, thereby providing purified renatured trypsinogen having reduced volume.

The “volume reduction step” reduces the volume of the renatured trypsinogen relative to the volume of the renatured trypsinogen prior to the volume reduction step. The volume may be reduced by at least ½, ¼, 1/10, 1/15, or 1/20 relative to the volume prior to the volume reduction step. Preferably, the volume reduction step reduces the volume of the renatured trypsinogen by at least 1/10 relative to the volume of the renatured trypsinogen prior to the volume reduction step.

In a preferable embodiment, the volume reduction step is carried out prior to a step of purifying renatured trypsinogen by anion exchange chromatography.

The method may further comprise a step of subjecting the renatured trypsinogen (e.g. prior to anion exchange chromatography) to a filtration step that removes molecules having a size of less than 20 kDa, preferably less than 15 kDa, more preferably less than 10 kDa. Additionally or alternatively, the method may further comprise a step of subjecting the purified renatured trypsinogen (e.g. subsequent to anion exchange chromatography) to a filtration step that removes molecules having a size of less than 20 kDa, preferably less than 15 kDa, more preferably less than 10 kDa.

The filtration step preferably has the effect of removing L-arginine from the renatured trypsinogen (e.g. prior to anion exchange chromatography) or the purified renatured trypsinogen (e.g. subsequent to anion exchange chromatography). Thus, the method may further comprise a step of subjecting the renatured trypsinogen (e.g. prior to anion exchange chromatography) to a filtration step that removes L-arginine. Additionally or alternatively, the method may further comprise a step of subjecting the purified renatured trypsinogen (e.g. subsequent to anion exchange chromatography) to a filtration step that removes L-arginine.

Additionally or alternatively, the filtration step may have the effect of removing GSSG or GSH.

In a preferable embodiment, the filtration step is carried out prior to a step of purifying renatured trypsinogen by anion exchange chromatography.

Said volume reduction step may be carried out prior to, simultaneously with or sequentially to said filtration step. For example, the renatured trypsinogen may be subjected to tangential flow filtration to simultaneously reduce the volume, e.g. provide the volume reduction step, and remove molecules having a size of less than 20 kDa (preferably less than 15 kDa, more preferably less than 10 kDa), e.g. the filtration step.

The “filtration step” encompasses any suitable means for removing molecules having a size as described herein (e.g. less than 20 kDa). For example, the filtration step may be carried out with a filter, for example a filter having a molecular weight cut-off of 20 kDa (preferably 15 kDa, more preferably 10 kDa). For example, the filter may retain molecules having a size of ≥20 kDa, preferably ≥15 kDa, more preferably ≥10 kDa.

Additionally or alternatively, the filtration step may be carried out by dialysis, for example by separating renatured trypsinogen from molecules having a size of less than 20 kDa (preferably less than 15 kDa, more preferably less than 10 kDa) based on the difference in their rates of diffusion through a semipermeable membrane, such as dialysis tubing.

A “denatured” trypsinogen is a trypsinogen which is not folded into the protein's native structure (e.g. secondary structure and/or tertiary), such that the trypsinogen does not have the basal level of proteolytic activity which folded trypsinogen has. Denatured trypsinogen may be provided by contacting trypsinogen with a denaturant, such as urea and/or a reducing agent such as 1,4-dithiothreitol (DTT).

Advantageously, denatured trypsinogen does not self-cleave (autolyse) during the purification steps of the invention, such that higher yields of trypsinogen (and thus β-trypsin) may be obtained.

Reference to “renaturing” the purified denatured trypsinogen means that the denatured trypsinogen is folded into the protein's native structure (e.g. secondary structure and/or tertiary, thus providing folded/renatured trypsinogen). Denatured trypsinogen may be renatured by contacting the denatured trypsinogen with an aggregation inhibitor such as arginine (e.g. L-arginine), oxidised glutathione (GSSG) and/or reduced glutathione (GSH), preferably L-arginine.

It is preferable to renature most or all of the denatured trypsinogen, to increase yields of renatured trypsinogen to be cleaved, thus providing increased yields of β-trypsin. The present inventors have uncovered particularly advantageous conditions under which the renaturation step may be carried out, which increase the yield of renatured trypsinogen.

In one embodiment, in the step of renaturing the purified denatured trypsinogen, L-arginine is present at a concentration of 0.6M to <1M. For example, L-arginine may preferably be present at a concentration of about 0.75M. The inventors have surprisingly found that L-arginine concentrations in this range (0.6M to <1M) is particularly advantageous for enhancing the yield of renatured trypsinogen (see FIG. 6A).

Additionally or alternatively, in the step of renaturing the purified denatured trypsinogen, denatured trypsinogen may be present at a concentration of <0.2 mg/mL. For example, denatured trypsinogen may be present at a concentration of >0.06 mg/ml to <0.2 mg/mL; preferably 0.08 mg/ml to 0.15 mg/ml; more preferably at a concentration of about 0.1 mg/mL. The inventors have surprisingly found that performing the renaturation step with a (starting) denatured trypsinogen concentration within such range (<0.2 mg/ml) is particularly advantageous for enhancing the yield of renatured trypsinogen (see FIG. 6B).

Such conditions (L-arginine and denatured trypsinogen concentration) may both be employed in combination to synergistically enhance the yield of renatured trypsinogen (see FIG. 6B).

Alternatively or additionally, denatured trypsinogen may be renatured by removing denaturant, for example removing denaturant employed during a step of solubilising the one or more insoluble inclusion bodies (e.g. step d)). Denaturant may be removed by filtration and/or diafiltration.

The term “producing denatured trypsinogen” means producing a denatured trypsinogen which is not comprised within an insoluble inclusion body. For example, “producing denatured trypsinogen” preferably comprises producing soluble (e.g. solubilised) denatured trypsinogen.

A particularly advantageous prokaryotic host cell which may be employed for forming such insoluble inclusion bodies is an Escherichia coli host cell. Thus, in one embodiment the prokaryotic host cells are Escherichia coli host cells (such as E. coli BL21 (DE3)).

It is known that trypsinogen can undergo autoactivation, a self-amplifying biomolecular reaction in which trypsinogen is autoactivated to provide trypsin, which in turn may activate a trypsinogen molecule to yield two trypsin molecules. Furthermore, it is known that this autocatalytic activity may be promoted, or on the other hand perturbed, depending on factors such as local pH, the presence or absence of metal ions and/or the presence or absence of trypsinogen inhibitors. By way of example (with regard to the latter) it is believed that inhibitors of certain proteolytic enzymes (e.g. trypsinogen) are present in the pancreas (where trypsinogen is produced) in order to suppress proteolytic activity, thus preventing pancreatic self-digestion which can lead to pancreatitis. Furthermore, it is believed that metal ion (in particular calcium) concentrations in the pancreas are kept below a threshold that would otherwise promote proteolytic activity of proteolytic enzymes such as trypsinogen and trypsin. Indeed, it has been demonstrated that hypercalcaemia (resulting in an abnormally high serum calcium concentration) may induce pancreatitis due to over-activation of proteolytic enzymes in the pancreas.

The term “conditions to promote proteolytic activity of the trypsinogen” means that the trypsinogen is incubated under conditions which are favourable to/stimulate the basal level of proteolytic activity in the trypsinogen such that it is self-cleaved/autolysed into trypsin, for example the incubation conditions may include the presence of an agent which accelerates (e.g. promotes) autolysis of trypsinogen. Thus, the term “promote” may be used synonymously with the terms “stimulate”, “induce” and/or “accelerate”.

Conditions that promote proteolytic activity of trypsinogen are well known in the art, and include incubation of trypsinogen in the presence of a metal ion. For example, it was shown that the presence of 50 mM calcium, Ca⁺⁺ (in 0.1M Tris-HCL buffer, pH 8.1) allowed for self-activation of trypsinogen even in the presence of a trypsin inhibitor (J Kay, B Kassell, J Biol Chem. 1971 November; 246(21):6661-5). The presence of other metal ions (such as strontium, barium, magnesium, sodium, lithium, potassium, ammonium, rubidium, caesium and neodymium) are also known to provide suitable conditions to promote proteolytic activity of trypsinogen.

The amino terminus of vertebrate trypsinogen contains the sequence Asp-Asp-Asp-Asp-Lys (SEQ ID NO.: 4, highly conserved during vertebrate evolution). Trypsinogen can autocleave (e.g. under conditions which promote proteolytic activity of the trypsinogen) the peptide bond between the lysine residue of said conserved sequence and the subsequent amino acid residue, typically isoleucine. The resulting N-terminal peptide, known as “trypsinogen activation peptide” is then released (see FIG. 3). Trypsinogen activation peptide (TAP) is a by-product of the trypsinogen (auto)activation process.

Autoactivated trypsinogen, lacking the trypsinogen activation peptide, may be referred to as “cleaved trypsinogen” herein (or alternatively, simply “trypsin”).

It may be confirmed that the conditions are suitable to promote proteolytic activity of the trypsinogen by detecting cleaved trypsinogen (lacking the trypsinogen activation peptide) following incubating the purified renatured trypsinogen under said conditions, optionally in the presence of a trypsin inhibitor e.g. at a concentration that suppresses trypsin activity.

Due to the removal of the trypsinogen activation peptide, the size of the activated trypsinogen molecule is reduced, typically from about 24 kDa to about 23.8 kDa. Thus, cleavage of the trypsinogen activation peptide may conveniently by detected by identifying a reduction in the size of the trypsinogen molecule, indicating a change from the inactive/zymogen form (comprising trypsinogen activation peptide) to the cleaved trypsinogen (e.g. trypsin) form that lacks trypsinogen activation peptide. Such reduction in size may be conveniently measured by SDS-PAGE analysis, or by chromatography (see FIG. 3A). A suitable chromatography technique includes high-performance liquid chromatography, for example, reverse-phase high-performance liquid chromatography.

Additionally or alternatively, the presence of (cleaved) trypsinogen activation peptide may be detected to confirm proteolytic activity of the trypsinogen, for example, by means of a suitable immunoassay (preferably ELISA) for specific detection of cleaved trypsinogen activation peptide. An example of a suitable, commercially available kit for performing an ELISA to quantify TAP is the “Bovine Trypsinogen Activation Peptide (TAP) ELISA Kit” (MyBioSource Inc., Catalogue No. MBS2609836).

The “conditions that promote the proteolytic activity of trypsinogen” may be conditions in which after 1 hour, at least 20% of the trypsinogen present in a sample has liberated (e.g. autocleaved) its trypsinogen activation peptide and thus been converted into trypsin. For example, conditions in which after 1 hour, at least 30%, 40%, 50%, 60%, 70%, 80% or 90% (preferably at least 40%) of the trypsinogen in a sample has liberated its trypsinogen activation peptide and thus been converted into trypsin. Preferably, the “conditions that promote proteolytic activity of trypsinogen” may be conditions in which after 1 hour, at least 50% of the trypsinogen in a sample has liberated (e.g. autocleaved) its trypsinogen activation peptide and thus been converted into trypsin. Said percentage (%) values are intended to be relative to baseline levels of trypsinogen, for example the level of trypsinogen at ≤1 min, ≤0.5 min or ≤0.1 min (preferably 0 min) following commencement of the step of incubating the purified renatured trypsinogen under conditions to promote proteolytic activity of the trypsinogen.

The “conditions that promote the proteolytic activity of trypsinogen” may be conditions in which after 1 hour, at least 20% of the trypsinogen present in a sample has liberated (e.g. autocleaved) its trypsinogen activation peptide and thus been converted into trypsin. For example, conditions in which after 5 hours, at least 30%, 40%, 50%, 60%, 70%, 80% or 90% (preferably at least 40%) of the trypsinogen in a sample has liberated its trypsinogen activation peptide and thus been converted into trypsin. Preferably, the “conditions that promote proteolytic activity of trypsinogen” may be conditions in which after 5 hours, at least 50% of the trypsinogen in a sample has liberated (e.g. autocleaved) its trypsinogen activation peptide and thus been converted into trypsin. Said percentage (%) values are intended to be relative to baseline levels of trypsinogen, for example the level of trypsinogen at ≤1 min, ≤0.5 min or ≤0.1 min (preferably 0 min) following commencement of the step of incubating the purified renatured trypsinogen under conditions to promote proteolytic activity of the trypsinogen.

The “conditions that promote the proteolytic activity of trypsinogen” may be conditions in which after 1 hour, at least 20% of the trypsinogen present in a sample has liberated (e.g. autocleaved) its trypsinogen activation peptide and thus been converted into trypsin. For example, conditions in which after 10 hours, at least 30%, 40%, 50%, 60%, 70%, 80% or 90% (preferably at least 40%) of the trypsinogen in a sample has liberated its trypsinogen activation peptide and thus been converted into trypsin. Preferably, the “conditions that promote proteolytic activity of trypsinogen” may be conditions in which after 5 hours, at least 50% of the trypsinogen in a sample has liberated (e.g. autocleaved) its trypsinogen activation peptide and thus been converted into trypsin. Said percentage (%) values are intended to be relative to baseline levels of trypsinogen, for example the level of trypsinogen at ≤1 min, ≤0.5 min or ≤0.1 min (preferably 0 min) following commencement of the step of incubating the purified renatured trypsinogen under conditions to promote proteolytic activity of the trypsinogen.

The “conditions that promote the proteolytic activity of trypsinogen” may be conditions in which after 10 hours, at least 20% of the trypsinogen present in a sample has liberated (e.g. autocleaved) its trypsinogen activation peptide and thus been converted into trypsin. For example, conditions in which after 15 hours, at least 30%, 40%, 50%, 60%, 70%, 80% or 90% (preferably at least 50%) of the trypsinogen in a sample has liberated its trypsinogen activation peptide and thus been converted into trypsin. Preferably, the “conditions that promote proteolytic activity of trypsinogen” may be conditions in which after 20 hours, at least 50% (preferably at least 80%) of the trypsinogen in a sample has liberated (e.g. autocleaved) its trypsinogen activation peptide and thus been converted into trypsin. Said percentage (%) values are intended to be relative to baseline levels of trypsinogen, for example the level of trypsinogen at ≤1 min, ≤0.5 min or ≤0.1 min (preferably 0 min) following commencement of the step of incubating the purified renatured trypsinogen under conditions to promote proteolytic activity of the trypsinogen.

Preferably, said determination is carried out in the presence of a trypsin inhibitor (e.g. at a concentration that suppresses trypsin activity). Examples of suitable trypsin inhibitors include diisopropyl fluorophosphate, 3,4-Dichloroisocoumarin serine protease inhibitor, Benzamidine hydrochloride, and 4-Amidinophenylmethanesulfonyl fluoride hydrochloride serine protease inhibitor. A preferable trypsin inhibitor is diisopropyl fluorophosphate, preferably at a concentration of 0.05-0.15 mM (more preferably about 0.10 mM).

In contrast, “conditions that do not promote the proteolytic activity of trypsinogen” may be conditions in which the trypsinogen has substantially no proteolytic activity. Thus, the “conditions that do not promote the proteolytic activity of trypsinogen” may be conditions in which after 1 hour, less than 20% of the trypsinogen present in a sample has liberated (e.g. autocleaved) its trypsinogen activation peptide and thus been converted into trypsin. For example, conditions in which after 1 hour, less than 15%, 10%, 5%, 1% or 0.01% of the trypsinogen in a sample has liberated its trypsinogen activation peptide and thus been converted into trypsin. Preferably, the “conditions that promote proteolytic activity of trypsinogen” may be conditions in which after 1 hour, 0% of the trypsinogen in a sample has liberated (e.g. autocleaved) its trypsinogen activation peptide and thus been converted into trypsin.

Proteolytic activity of the trypsinogen may be promoted (e.g. accelerated) by incubating the trypsinogen in the presence a suitable metal ion, such as an alkali metal ion, alkaline earth metal ion and/or lanthanide ion. Specific examples of suitable metal ions include cations of calcium, strontium, barium, magnesium, sodium, lithium, potassium, ammonium, rubidium, caesium and/or neodymium. For ease of use, the cation may be associated with an appropriate anion, such as a sulphate, citrate, acetate, chloride, and/or fluoride anion. The metal ion (e.g. cation) may be added (e.g. in a step of incubating the purified renatured trypsinogen under conditions to promote proteolytic activity of the trypsinogen) to a final concentration of at least 10 mM; more preferably at least 20 mM. For example, the metal ion may be added to a final concentration of 20-150 mM. In a preferred embodiment, the metal ion is added to a final concentration of 50-100 mM.

In one embodiment the proteolytic activity of the renatured trypsinogen and/or trypsinogen is promoted by adding calcium (e.g. Ca⁺⁺).

In one embodiment, in the step of incubating the purified renatured trypsinogen under conditions to promote proteolytic activity of the trypsinogen (e.g. step c. of the first aspect, step h. of the second aspect), calcium may be added to a final concentration of at least 10 mM, preferably at least 20 mM. For example, in said step, calcium may be added to a final concentration of 20-150 mM, preferably 50-100 mM.

In one embodiment, in the step of incubating the purified renatured trypsinogen under conditions to promote proteolytic activity of the trypsinogen (e.g. step c. of the first aspect, step h. of the second aspect), the sample is incubated for 5-48 hours, preferably for 5-20 hours. The sample may be incubated (in step c)) at a temperature of 15-30° C. (preferably about 25° C.). Advantageously, such temperature may promote the catalytic activity of trypsin/trypsinogen.

Additionally or alternatively, such conditions which promote proteolytic activity of the trypsinogen may include the presence of an optimal pH which is favourable to trypsinogen autolysis, and thus stimulates the basal level proteolytic activity in the trypsinogen. Thus, the pH may be adjusted in an appropriate manner to promote said proteolytic activity.

By promoting the basal level of trypsinogen for autolysis (such that trypsinogen converts itself into fully active trypsin), the resulting trypsin can further contribute to proteolytic cleavage of the remaining trypsinogen, allowing for efficient production of trypsin. Advantageously, this avoids the need to provide exogenous trypsin (or other non-trypsinogen proteolytic peptide) to promote/catalyse activation, which may be of low purity and/or having contaminants which compromise the GMP status of the method.

In a preferable embodiment, the proteolytic activity of the renatured trypsinogen is promoted (e.g. stimulated) by adding calcium.

The calcium may be added (in a step of incubating the purified renatured trypsinogen under conditions to promote proteolytic activity of the trypsinogen e.g. in step h) to a final concentration of at least 10 mM; more preferably at least 20 mM. For example, the calcium may added (in a step of incubating the purified renatured trypsinogen under conditions to promote proteolytic activity of the trypsinogen e.g. in step h) to a final concentration of 20-150 mM.

In a preferred embodiment, the calcium is added (in a step of incubating the purified renatured trypsinogen under conditions to promote proteolytic activity of the trypsinogen e.g. in step h) to a final concentration of 50-100 mM.

The inventors have identified advantageous incubation times and temperatures for the activation step (e.g. step h), e.g. which allow good yield of activated β-trypsin, but without “over activation” which may result in auto-cleavage of the β-trypsin (e.g. into less preferable α-trypsin).

In one embodiment, in a step of incubating the purified renatured trypsinogen under conditions to promote proteolytic activity of the trypsinogen (e.g. step h) (the activation step), the sample is incubated for 5-48 hours; preferably for 5-20 hours.

In a step of incubating the purified renatured trypsinogen under conditions to promote proteolytic activity of the trypsinogen (e.g. step h)), the sample may be incubated at a temperature of 15-30° C. (for example about 25° C.).

To ensure that trypsinogen does not exhibit proteolytic activity during purification, the method steps may advantageously be carried out under conditions that do not promote the proteolytic activity of the (renatured) trypsinogen.

In one embodiment (e.g. of said first aspect), steps a) and b) are carried out in the absence of a metal ion that promotes proteolytic activity (e.g. such that calcium is not available to promote proteolytic activity). Additionally or alternatively, steps ii), iii), iv) and/or v) (e.g. of said first aspect) are carried out in the absence of such metal ion. Such metal ions include cations of calcium, strontium, barium, magnesium, sodium, lithium, potassium, ammonium, rubidium, caesium and neodymium.

In one embodiment (e.g. of the second aspect), steps f) and g) are carried out in the absence of a metal ion that promotes proteolytic activity (e.g. such that calcium is not available to promote proteolytic activity). Additionally or alternatively, steps b), c), d), and/or e) (e.g. of the second aspect) are carried out in the absence of such metal ion. Such metal ions include cations of calcium, strontium, barium, magnesium, sodium, lithium, potassium, ammonium, rubidium, caesium and neodymium.

The term “the absence of a metal ion that promotes proteolytic activity” means that there is substantially no such metal ion (e.g. the concentration of such metal ion is below a threshold required to promote proteolytic activity). Reference to “substantially no such metal ion” may mean a metal ion concentration of ≤2 mM, ≤1 mM, or ≤0.5 mM; preferably ≤0.1 mM. In a more preferred embodiment, “substantially no metal ion” means that there is no such metal ion present.

In one embodiment (e.g. of the first aspect), steps a) and b) are carried out in the absence of calcium (e.g. such that calcium is not available to promote proteolytic activity). Additionally or alternatively, steps ii), iii), iv), and/or v) (e.g. of the first aspect) are carried out in the absence of calcium.

In a preferable embodiment (e.g. of the second aspect), steps a), b), ii), iii), iv), and/or v) are carried out in the absence of calcium. For example, each of steps a), b), ii), iii), iv), and v) (e.g. of the second aspect) may be carried out in the absence of calcium.

In one embodiment (e.g. of the second aspect), steps f) and g) are carried out in the absence of calcium (e.g. such that calcium is not available to promote proteolytic activity). Additionally or alternatively, steps b), c), d), and/or e) (e.g. of the second aspect) are carried out in the absence of calcium.

In a preferable embodiment (e.g. of the second aspect), steps b), c), d), e), f) and/or g) are carried out in the absence of calcium. For example, each of steps b), c), d), e), f) and g) may be carried out in the absence of calcium.

The term “the absence of calcium” means that there is substantially no calcium (e.g. the concentration of calcium is below a threshold required to promote proteolytic activity). Reference to “substantially no calcium” may mean a calcium concentration of ≤2 mM, ≤1 mM, or ≤0.5 mM; preferably ≤0.1 mM. In a more preferred embodiment, “substantially no calcium” means that there is no calcium present.

Suitably, step b) and/or step c) (e.g. of the first aspect) may be carried out at a temperature of 1-10° C.; preferably 2-8° C. Suitably, step ii) and/or step iii) (e.g. of the second aspect) may be carried out at a temperature of 1-10° C.; preferably 2-8° C.

The present inventors have found that a β-trypsin preparation produced by a method of the invention is of higher purity than prior art β-trypsin preparations and exhibits higher proteolytic activity levels.

In another aspect, the invention provides a β-trypsin composition (e.g. obtainable by a method described herein), wherein at least 80% of the total polypeptides comprised in the composition are β-trypsin and wherein the composition has an activity level of at least 3000 USP units/mg of total polypeptides, optionally wherein the activity level is determined by an assay comprising:
a) admixing 0.075 mL of the β-trypsin composition (e.g. test sample) with 3.125 ml of reaction buffer comprising (or consisting of) substrate N_{α}-Benzoyl-L-arginine ethyl ester (BAEE), sodium phosphate and hydrochloric acid, the reaction buffer having a temperature of 25° C., to provide an admixture having:
   i. 0.25 mM BAEE;
   ii. 67 mM sodium phosphate buffer;
   iii. 0.031-0.063 mM hydrochloric acid;
   iv. a pH of 7.6 at 25° C.; and
   v. a volume of 3.2 mL;
b) incubating for 4 minutes at 25° C.;
c) measuring an amount of cleavage of substrate BAEE by detecting absorbance at 253 nm (A₂₅₃) of the admixture immediately after preparing the admixture and after incubation at step b); and calculating the BAEE units (U)/ml of enzyme in the β-trypsin composition using the following formula:

$$Activity BAEE U / ml = \frac{( \frac{{A}_{2} - {A}_{1}}{t} ) \times D F}{{A}_{253 / min} \times V}$$

wherein: A₁ is the A₂₅₃ immediately after preparing the admixture;
   A₂ is the A₂₅₃ of the admixture after 4 minutes of incubation,
   t is the duration of the incubation;
   DF is the dilution factor;
   A_{253/min} is 0.001; and
   V is the volume of the β-trypsin composition in the admixture, 0.075 mL; and
converting BAEE units/ml to USP units/mg by dividing the BAEE U/ml by the concentration of the β-trypsin composition (e.g. mg/ml of polypeptide) and subsequently dividing by three.

In another aspect, the invention provides a β-trypsin composition (e.g. obtainable by a method described herein), wherein at least 80% of the total polypeptides comprised in the composition are β-trypsin and wherein the composition has an activity level of at least 3000 USP units/mg of total polypeptides, optionally wherein the activity level is determined by an assay comprising:
a) admixing 0.075 mL of the β-trypsin composition (e.g. test sample) with 3.125 ml of reaction buffer comprising (or consisting of) substrate N_{α}-Benzoyl-L-arginine ethyl ester (BAEE), sodium phosphate and hydrochloric acid, the reaction buffer having a temperature of 25° C., to provide an admixture having:
   i. 0.25 mM BAEE;
   ii. 67 mM sodium phosphate buffer;
   iii. 0.031-0.063 mM hydrochloric acid;
   iv. a pH of 7.6 at 25° C.; and
   v. a volume of 3.2 mL;
b) incubating for 5 minutes at 25° C.;
c) measuring an amount of cleavage of substrate BAEE by detecting absorbance at 253 nm (A₂₅₃) of the admixture immediately after preparing the admixture and after incubation at step b); and calculating the BAEE units (U)/ml of enzyme in the β-trypsin composition using the following formula:

$$Activity BAEE U / ml = \frac{( \frac{{A}_{2} - {A}_{1}}{t} ) \times D F}{{A}_{253 / min} \times V}$$

wherein: A₁ is the A₂₅₃ immediately after preparing the admixture;
   A₂ is the A₂₅₃ of the admixture after incubation,
   t is the duration of the incubation;
   DF is the dilution factor;
   A_{253/min} is 0.001; and
   V is the volume of the β-trypsin composition in the admixture, 0.075 mL; and
converting BAEE units/ml to USP units/mg by dividing the BAEE U/ml by the concentration of the β-trypsin composition (e.g. mg/ml of polypeptide) and subsequently dividing by three.

In another aspect, the invention provides a β-trypsin composition (e.g. obtainable by a method described herein), wherein at least 80% of the total polypeptides comprised in the composition are β-trypsin and wherein the composition has an activity level of at least 3000 USP units/mg of total polypeptides, optionally wherein the activity level is determined by an assay comprising:
a) admixing 0.075 ml of the β-trypsin composition (e.g. test sample) with 3.125 ml of reaction buffer comprising (or consisting of) substrate N_{α}-Benzoyl-L-arginine ethyl ester (BAEE), sodium phosphate and hydrochloric acid, the reaction buffer having a temperature of 25° C., to provide an admixture having:
   i. 0.075 ml of the β-trypsin composition;
   ii. 0.25 mM BAEE;
   iii. 67 mM sodium phosphate buffer;
   iv. 0.031-0.063 mM hydrochloric acid;
   v. a pH of 7.6 at 25° C.; and
   vi. a volume of 3.2 ml;
b) providing a control sample with which the β-trypsin composition is not admixed, the control sample comprising (or consisting of):
   i. 0.25 mM BAEE;
   ii. 67 mM sodium phosphate buffer;
   iii. 0.031-0.063 mM hydrochloric acid;
   iv. a pH of 7.6 at 25° C.;
   v. a volume of 3.2 ml; and
   vi. no β-trypsin composition;
c) incubating the admixture of step a) and the control sample of step b) for 5 minutes at 25° C.;
d) measuring an amount of cleavage of substrate BAEE by: detecting absorbance at 253 nm (A₂₅₃) of the admixture of step a) every minute for 5 minutes; detecting absorbance at 253 nm (A₂₅₃) of the control sample of step b) every minute for 5 minutes; obtaining the ΔA₂₅₃/minute using the maximum linear rate for the admixture of step a) and the control sample of step b); and calculating the BAEE units (U)/ml of enzyme in the β-trypsin composition using the following formula:

$$BAEE units / ml enzyme = \frac{( Δ {A}_{253} / minute Test - Δ {A}_{253} / minute Blank ) \times ( df )}{( 0.001 ) \times ( 0.075 )}$$

wherein: df is the dilution factor;
   0.001=the change in A₂₅₃/minute based on unit definition;
   0.075 ml=the volume of the β-trypsin composition in the admixture;
converting BAEE units/ml to USP units/mg by dividing the BAEE U/ml by the concentration of the β-trypsin composition (e.g. mg/ml of polypeptide) and subsequently dividing by three.

The term “immediately after preparing the admixture” may mean within ≤15 seconds, ≤10 seconds, ≤5 seconds, or ≤1 second after preparing the admixture.

When measuring absorbance A₂₅₃ (e.g. with a spectrophotometer), the light path is preferably 1 cm. When measuring absorbance A₂₅₃ (e.g. with a spectrophotometer), the volume in the vessel (e.g. cuvette) comprising the sample to be measured is preferably 3.2 ml.

One BAEE unit of trypsin activity produces a change (e.g. increase) in A₂₅₃ of 0.001 per minute with BAEE as substrate at pH 7.6 at 25° C. in a reaction volume of 3.20 ml. Thus, 0.001 is the change in A₂₅₃/minute based on unit definition.

A dilution factor (DF) is used, as the A₂₅₃ is typically detected by diluting an aliquot of the admixture in a larger volume (e.g. of buffer) to provide a diluted sample, with the A₂₅₃ being measured in said diluted sample. For example, where a 10 μl aliquot of the admixture is diluted in a volume of 1 mL, the dilution factor is 100.

Said β-trypsin composition (e.g. obtainable by a method described herein) may have an activity level of at least 3500 USP units/mg, 4000 USP units/mg, 4500 USP units/mg, or 5000 USP units/mg of total polypeptides.

In one embodiment, at least 85% (preferably at least 90%; more preferably at least 95%) of the total polypeptides comprised in the composition are β-trypsin.

A β-trypsin produced by a method of the invention is particularly advantageous for activating a clostridial neurotoxin (e.g. a BoNT, such as BoNT/E) by cleaving a single-chain clostridial neurotoxin to produce an active di-chain clostridial neurotoxin.

In one embodiment, a method of the invention further comprises contacting a single-chain clostridial neurotoxin having an activation loop with the β-trypsin (e.g. the β-trypsin produced by a method described herein), wherein the β-trypsin hydrolyses a peptide bond of the activation loop of the single-chain clostridial neurotoxin, thereby producing a di-chain clostridial neurotoxin.

One aspect of the invention provides a method for producing a di-chain clostridial neurotoxin, the method comprising:
a. providing a single-chain clostridial neurotoxin having an activation loop; and
b. contacting the single-chain clostridial neurotoxin with the β-trypsin composition disclosed herein (e.g. obtainable by a method described herein);
c. wherein the β-trypsin hydrolyses a peptide bond of the activation loop of the single-chain clostridial neurotoxin, thereby producing a di-chain clostridial neurotoxin.

The invention further embraces a di-chain clostridial neurotoxin obtainable by such method (the method for producing a di-chain clostridial neurotoxin disclosed herein).

Preferably, said clostridial neurotoxin is a botulinum neurotoxin. Examples of suitable botulinum neurotoxin serotypes include A, B, C1, D, E, F and G. In a preferable embodiment, the botulinum neurotoxin is botulinum neurotoxin serotype E (BoNT/E).

Botulinum neurotoxin (BoNT) is produced by C. botulinum in the form of a large protein complex, consisting of BoNT itself complexed to a number of accessory proteins. There are seven different classes of botulinum neurotoxin, namely: botulinum neurotoxin serotypes A, B, C1, D, E, F and G, all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises a C-terminal targeting component (H_{C} domain) and an N-terminal translocation component (H_{N} domain). The cleavage site is located between the L-chain and the translocation components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease). Non-cytotoxic proteases act by proteolytically-cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

When a single-chain BoNT/E1 protein (for example) is contacted with trypsin, the proteolytic action of trypsin cleaves the single-chain protein at a site between the L chain protease component and the translocation component to produce a di-chain protein, where the two chains are linked by a disulphide bridge. In more detail, the two chains formed following cleavage of single-chain BoNT/E1 at the activation site are a first chain of amino acid residues 1-419 and a second chain of amino acid residues 423-1252, with residues 420, 421 and 422 removed by the cleavage event. Thus, β-trypsin can be used to activate the single-chain polypeptide by converting it to the active di-chain form. Thus, advantageously, the use of β-trypsin means that it is not necessary to engineer an exogenous (non-native) cleavage site into a BoNT/E1 of the invention.

An exemplary L-chain reference sequence of BoNT/E includes amino acid residues 1-422 of BoNT/E. However, this reference sequence should be considered a guide, as slight variations may occur according to sub-serotypes. By way of example, US 2007/0166332 (hereby incorporated by reference in its entirety) cites a slightly different BoNT/E L-chain sequence of amino acid residues M1-R422.

Examples of the BoNT/E H_{C} domain reference sequence includes amino acid residues R846-K1252.

| The L-chain of BoNT/E may be as follows (SEQ ID NO.: 2): | |
|---|---|
| 1 | PKINSFNYND PVNDRTILYI KPGGCQEFYK SFNIMKNIWI IPERNVIGTT |
| 51 | PQDFHPPTSL KNGDSSYYDP NYLOSDEEKD RFLKIVIKIF NRINNNLSGG |
| 101 | ILLEELSKAN PYLGNDNTPD NQFHIGDASA VEIKFSNGSQ DILLPNVIIM |
| 151 | GAEPDLFEIN SSNISLRNNY AIVTFSPEYS FRENDNSMNE FRFNDNSMNE |
| 201 | FIQDPALTLM HELIHSLHGL YGAKGITTKY TITQKQNPLI TNIRGTNIEE |
| 251 | FLTFGGTDLN IITSAQSNDI YTNLLADYKK IASKLSKVQV SNPLLNPYKD |
| 301 | VFEAKYGLDK DASGIYSVNI NKFNDIFKKL YSFTEFDLAT KFQVKCRQTY |
| 351 | IGQYKYFKLS NLLNDSIYNI SEGYNINNLK VNFRGQNANL NPRIITPITG |
| 401 | RGLVKKIIRF CKNIVSVKGI R |
| The H-chain of BoNT/E may be as follows (SEQ ID NO.: 3): | |
| 1 | KSICIEINNG ELFFVASENS YNDDNINTPK EIDDIVISNN NYENDLDQVI |
| 51 | LNFNSESAPG LSDEKLNLTI QNDAYIPKYD SNGTSDIEQH DVNELNVFFY |
| 101 | LDAQKVPEGE NNVNLISSID TALLEQPKIY TFFSSEFINN VNKPVQAALF |
| 151 | VSWIQQVLVD FTTEANQKST VDKIADISIV VPYIGLALNI GNEAQKGNFK |
| 201 | DALELLGAGI LLEFEPELLI PTILVFTIKS FLGSSDNKNK VIKAINNALK |
| 251 | ERDEKWKEVY SFIVSNWMTK INTQFNKRKE QMYQALQNQV NAIKTIIESK |
| 301 | YNSYTLEEKN ELTNKYDIKQ IENELNQKVS IAMNNIDRFL TESSISYLMK |
| 351 | LINEVKINKL REYDENVKTY LLNYIIQHGS ILGESQQELN SMVTDTLNNS |
| 401 | IPFKLSSYTD DKILISYENK FFKRIKSSSV LNMRYKNDKY VDISGYDSNI |
| 451 | NINGDVYKYP TNKNOFGIYN DKLSEVNISQ NDYIIYDNKY KNFSISFWVR |
| 501 | IPNYDNKIVN VNNEYTIINC MRDNNSGWKV SLNHNEIIWT LQDNAGINQK |
| 551 | LAFNYGNANG ISDYINKWIF VTITNDRLGD SKLYINGNLI DQKSILNLGN |
| 601 | IHVSDNILFK IVNCSYTRYI GIRYFNIFDK ELDETEIQTL YSNEPNTNIL |
| 651 | KDFWGNYLLY DKEYYLLNVL KPNNFIDRRK DSTLSINNIR STILLANRLY |
| 701 | SGIKVKIQRV NNSSINDNLV RKNDQVYINF VASKTHLFPL YADTATTNKE |
| 751 | KTIKISSSGN RFNQVVVMNS VGNNCIMNFK NNNGNNIGLL GFKADTVVAS |
| 801 | TWYYTHMRDH TNSNGCFWNF ISEEHGWQEK |

The H_{C} domain of BoNT/E comprises two distinct structural features that are referred to as the H_{CC} and H_{CN} domains. Amino acid residues involved in receptor binding are believed to be primarily located in the H_{CC} domain. An example of the BoNT/E H_{CN} domain reference sequence includes amino acid residues 846-1085.

The above sequence positions may vary slightly according to sub-type, and further examples of suitable (reference) BoNT/E H_{CN} domains includes amino acid residues 848-1085.

BoNT/E may be produced by C. botulinum or C. butyricum, preferably C. botulinum. In one embodiment, the BoNT/E is produced in a non-clostridial cell. Alternatively (preferably) BoNT/E may be produced in a recombinant form (e.g. in Escherichia coli). Thus in one embodiment, BoNT/E for use in the invention is produced in a heterologous expression system, such as an E. coli. In one embodiment, the E. coli cell is E. coli BLR (DE3).

Further details on BoNT/E produced in a heterologous expression system (such as an E. coli) are described in WO 2014/068317 A1, which is incorporated herein by reference.

In one embodiment, reference to β-trypsin embraces trypsin-like enzymes that cleave at the same protease cleavage site as trypsin.

Trypsin cleaves protein sequences in which particular amino acids lie at certain positions on either side of the cleaved peptide bond. Such sequences can be represented by the nomenclature P4-P3-P2-P1-cleaved bond-P′1-P′2-P′3-P′4; in which P1 to P4 designate amino acids positioned 1 to 4 positions to the N-terminal side of the cleaved peptide bond respectively and P′1 to P′4 designate 1 to 4 positions C-terminal of the cleaved peptide bond, respectively.

Most particularly, trypsin cleaves protein sequences where either Arg or Lys amino acids occupy the P1 position. When Lys is in the P1 position there are three major types of sequence that are not sensitive to trypsin:
(1) Pro in the P′1 position usually reduces susceptibility to cleavage by trypsin (but not when Trp is in position P2);
(2) Either Cys or Asp in the P2 position together with Asp in the P′1 position reduces susceptibility to cleavage by trypsin; and
(3) Cys in the P2 position together with either His or Try in the P′1 position reduces susceptibility to cleavage by trypsin.

When Arg is in the P1 position there are also three major types of sequence that are not sensitive to trypsin:
(1) Pro in the P′1 position usually reduces susceptibility to cleavage by trypsin (but not when either Met, or possibly Glu, is in position P2);
(2) Cys in the P2 position together with Lys in the P′1 position reduces susceptibility to cleavage by trypsin; and
(3) Arg in the P2 position together with either His or Arg in the P′1 position reduces susceptibility to cleavage by trypsin.

Methods of producing a di-chain clostridial neurotoxin may further include a step of separating the di-chain clostridial neurotoxin from the β-trypsin.

Suitable methodology is described in WO2014/068317, which is incorporated herein by reference. For example, the methods may further comprise separating the di-chain clostridial neurotoxin protein from the β-trypsin by contacting the solution containing di-chain clostridial neurotoxin protein and β-trypsin with a hydrophobic surface, wherein the di-chain clostridial neurotoxin protein preferentially binds to the hydrophobic surface.

Trypsinogen is an inactive zymogen which is converted into trypsin by cleavage and removal of a leader sequence. For example, trypsinogen may have a leader comprising the sequence Val(Asp)₄Lys (SEQ ID NO.: 5), present at the N-terminus of the protein. Examples of a suitable trypsinogen include bovine, human, porcine, ovine and/or murine trypsinogen.

In one embodiment, the trypsinogen is a wild-type trypsinogen (and thus, the β-trypsin is a wild-type trypsin).

In a preferable embodiment, the trypsinogen (and thus, also the β-trypsin) is bovine trypsinogen. Thus, a prokaryotic host cell of a method described herein may comprise a nucleotide sequence encoding a bovine trypsinogen.

A nucleotide sequence encoding a bovine trypsinogen may have at least 70% sequence identity to SEQ ID NO: 1. In one embodiment, a nucleotide sequence encoding a bovine trypsinogen may have at least 80% or 90% sequence identity to SEQ ID NO: 1.

A nucleotide sequence encoding a bovine trypsinogen may have at least 70% sequence identity to SEQ ID NO: 1, preferably with the proviso that nucleotides 1-45 at the 5′ end of the sequence correspond to nucleotides 1-45 at the 5′ end of SEQ ID NO: 1. In one embodiment, a nucleotide sequence encoding a bovine trypsinogen may have at least 80% or 90% sequence identity to SEQ ID NO: 1, preferably with the proviso that nucleotides 1-45 at the 5′ end of the sequence correspond to nucleotides 1-45 at the 5′ end of SEQ ID NO: 1.

Preferably, a nucleotide sequence encoding a bovine trypsinogen may comprise (or consist of) the sequence of SEQ ID NO: 1.

The trypsinogen sequence may further comprise, or be fused to, a sequence for promoting the purification of the recombinant protein. For example, the trypsinogen sequence may comprise a His-tag (polyhistidine tag) which is an amino acid motif having at least four (preferably at least six) contiguous histidine residues. Such His-tag may suitably be used for affinity purification of the tagged trypsinogen.

Trypsin is a serine protease which catalyses the cleavage of a peptide bond on the carboxy-terminus of basic amino acid residues such as lysine and arginine. Trypsin proteins are classified as EC 3.4.21.4. Detailed mechanisms of the catalytic hydrolysis of peptide (and ester) substrates by serine proteases have been established and are known in the art, and trypsin is a particularly well understood member of this class of enzymes. A preferred form of trypsin is β-trypsin, also known as the native form of trypsin provided subsequent to trypsinogen activation. Autolysis of β-trypsin (which may be cleaved at Lys131-Ser132 in the bovine sequence) results in α-trypsin which is held together by disulfide bridges.

Trypsin is autocatalytic, and thus cleaves itself (e.g. into α-trypsin and/or into inactive peptides). Advantageously, an inhibitor of proteolytic activity (e.g. calcium-mediated proteolytic activity) may be added once trypsin has been produced, to prevent further cleavage.

In one embodiment, a method of the invention may further comprise adding a chelator (which can bind and sequester a metal ion) to inhibit proteolytic activity of the trypsinogen and/or β-trypsin. Advantageously, this may mitigate autolysis of β-trypsin once the β-trypsin has been produced, by preventing metal ion (e.g. calcium) mediated promotion of proteolytic activity. For example, addition of a chelator (such as a calcium chelator) may be used to halt/quench the proteolytic activity that is promoted in step c) of the first aspect, and step h) of the second aspect.

Preferably said chelator is a calcium chelator.

Preferred chelators include EDTA, HEDTA, EDG, EDDS, GLDA, MGDA, isomers thereof, or combinations thereof, in particular EDTA.

Further purification (or “polishing”) steps may be undertaken to yet further improve the level of purity of the β-trypsin product. For example, the inventors have found that subjecting the β-trypsin to an affinity chromatography step can be used to separate β-trypsin from α-trypsin.

In one embodiment, a method of the invention (e.g. the second aspect) may further comprise isolating the β-trypsin.

In one embodiment, the β-trypsin is isolated by affinity chromatography, preferably benzamidine affinity chromatography.

An “inducible promoter” is a nucleic acid sequence which promotes transcription of a nucleotide sequence (e.g. gene) which is operably linked to the promoter, once the activity of the promoter has been induced. An inducible promoter can be regulated (induced) upon addition of a suitable inducing factor. Thus, an inducible promoter may be used to regulate the transcription/expression of an operably linked gene by switching the promoter from an “off” state to an “on” state by the introduction of an inducing factor. A suitable inducing factor is isopropyl β-D-1-thiogalactopyranoside (IPTG).

Through being “operably linked” to the inducible promoter, the transcription of a nucleotide sequence (e.g. gene) operably linked to the inducible promoter is under control of the inducible promoter, such that the nucleotide sequence will be transcribed upon introduction of the inducing factor (switching the promoter from an “off” state to an “on” state).

Advantageously, by inducing expression (as per step b)), a ‘burst’ of transcription and expression may be provided, which results in an increased fraction of the expressed trypsinogen being present in an insoluble inclusion body.

In one embodiment, expression of the trypsinogen is induced by adding IPTG. Preferably, the IPTG is added to provide a final concentration of 0.25-0.75 mM, more preferably about 0.5 mM.

Preferably the expression of the trypsinogen is induced for at least 4 hours; more preferably the expression of the trypsinogen is induced for at least 6 hours.

In one embodiment, expression of the trypsinogen is induced for at least 4-12 hours. For example, expression of the trypsinogen may be induced for at least 6-10 hours.

In a particularly preferred embodiment, expression of the trypsinogen is induced for about 8 hours.

The inventors have found that it is advantageous to monitor the growth of the culture, and have found that purifying the β-trypsin from cultures of a particular growth stage (e.g. as measured by optical density) provides improved yields.

In one embodiment, the host cells are cultured to an optical density at 600 nm (OD₆₀₀) of 35-65; or more preferably to an OD (OD₆₀₀) of 40-60.

One or more of the method steps described herein may be following by a filtration step, preferably a diafiltration step.

For example, the renatured trypsinogen may be subjected to filtration (e.g. diafiltration) subsequent to the step of renaturing the purified denatured trypsinogen (e.g. step f), thereby producing renatured trypsinogen) of the presently described method for producing β-trypsin.

In one embodiment, the β-trypsin (produced by a method of the invention) may be subjected to a filtration (e.g. diafiltration) step. For example, wherein the method further comprises isolating the β-trypsin (e.g. by affinity chromatography), the purified β-trypsin may be subjected to a filtration (e.g. diafiltration) step.

Said filtration preferably comprises subjecting the renatured trypsinogen, β-trypsin and/or affinity chromatography-purified β-trypsin to a filtration step that removes molecules having a size less than about 0.2 microns.

Additionally or alternatively, said filtration step may comprise tangential flow filtration, employing, for example, a 0.5 m² or 0.2 m² cassette.

Furthermore, the inventors have deciphered particularly suitable operating conditions for a number of the method steps described herein, which may optimise the purification protocol resulting in further improvements in β-trypsin yield and purity. Examples of such optimised conditions are outlined below, where reference to a step means the corresponding step in a method of the second aspect described herein. Details of the buffers referred to are provided in the Examples.

For example, a step of isolating the one or more insoluble inclusion bodies from the host cells (e.g. step c)) may comprise one or more (or all) of the following steps: providing a bacterial pellet from the culture of prokaryotic cells (e.g. by subjecting the culture of prokaryotic cells to centrifugation); resuspending the pellet in a suitable buffer, such as SM-E01 buffer (described in the Examples); lysing the bacterial cells to provide a lysate, for example by homogenisation, preferably at a pressure of 650-750 bar; providing a pellet from the lysate (e.g. by subjecting the lysate to centrifugation); washing the lysate, preferably via resuspension of the lysate in SM-F01 and/or SM-F02 buffer (described in the Examples). The temperature during one or more (preferably all) of said steps may be 2-8° C.

A step of solubilising the one or more insoluble inclusion bodies, thereby producing denatured trypsinogen (e.g. step d)) may comprise one or more (or all) of the following steps: resuspending IBs in a suitable buffer, such as S-F01 buffer (described in the Examples), for example via homogenisation (e.g. for 12-15 hours, with agitation at 250-400 rpm) to provide solubilised denatured trypsinogen; subjecting the solubilised denatured trypsinogen to centrifugation (e.g. at 700-900 rpm for 50-50 mins) and retaining the supernatant; subjecting the supernatant to filtration, for example with a filter having a 0.2 μm pore size. The temperature during one or more (or all) of said steps may be 2-8° C.

A step of purifying the denatured trypsinogen by cation exchange chromatography, thereby providing purified denatured trypsinogen (e.g. step e)) may comprise one or more (or all) of the following steps: equilibrating a column with a suitable buffer (such as S-F02 buffer, described in the Examples), preferably wherein the column has a volume of 1492-1649 mL and/or a bed height of 15-25 cm (preferably 20 cm) and/or is packed with an Eshmuno S resin; loading the denatured trypsinogen on the column; washing the column with a suitable buffer (such as S-F02 buffer, described in the Examples); eluting the denatured trypsinogen from the column with a suitable buffer (such as S-G01 buffer, described in the Examples), to provide purified denatured trypsinogen.

A step of renaturing the purified denatured trypsinogen, thereby producing renatured trypsinogen (e.g. step f)) may comprise one or more (or all) of the following steps: adding a suitable buffer (such as S-H01 buffer, described in the Examples) to the purified denatured trypsinogen, to provide a concentration of purified denatured trypsinogen of 0.05-0.5 mg/ml (preferably about 0.1 mg/ml); agitating the solution, for example at 200 rpm; incubating the solution for 48-168 hours; subjecting the solution to filtration, for example with a filter having a 0.2 μm and/or 0.5 μm pore size. The temperature during one or more (or all) of said steps may be 2-8° C.

A step of purifying the renatured trypsinogen by anion exchange chromatography, thereby providing purified renatured trypsinogen (e.g. step g)) may comprise one or more (or all) of the following steps: equilibrating a column with a suitable buffer (such as S-H02 buffer, described in the Examples), preferably wherein the column has a volume of 177-216 mL and/or a bed height of 5-15 cm (preferably 10 cm) and/or is packed with an Eshmuno Q resin; loading the renatured trypsinogen on the column; collecting the flow-through, to provide purified renatured trypsinogen; washing the column with a suitable buffer (such as S-H02 buffer, described in the Examples). The temperature during one or more (or all) of said steps may be 2-8° C.

A step of incubating the purified renatured trypsinogen under conditions to promote proteolytic activity of the trypsinogen, wherein trypsinogen is cleaved into β-trypsin by said proteolytic activity (e.g. step h)) may comprise one or more (or all) of the following steps: incubating the purified renatured trypsinogen in the presence of calcium (e.g. S12 buffer, described in the Examples), wherein the calcium is present at a concentration of 60-90 mM (preferably about 75 mM); adding a calcium chelator (e.g. S-K01 buffer, described in the Examples), and preferably adjusting the pH to 7.2-7.6 (e.g. with S-01 buffer, described in the Examples); subjecting the solution to filtration, for example with a filter having a 0.2 μm and/or 0.5 μm pore size. Said incubation may be carried out a temperature of 20-25° C.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

A R N D C Q E G H I L K M F P S T W Y V
A 4
R −1 5
N -2 0 6
D -2 -2 1 6
C 0 -3 -3 -3 9
Q -1 1 0 0 -3 5
E -1 0 0 2 -4 2 5
G 0 -2 0 -1 -3 -2 -2 6
H -2 0 1 -1 -3 0 0 -2 8
I -1 -3 -3 -3 -1 -3 -3 -4 -3 4
L -1 -2 -3 -4 -1 -2 -3 -4 -3 2 4
K -1 2 0-1 -3 1 1 -2 -1 -3 -2 5
M -1 -1 -2 -3 -1 0 -2 -3 -2 1 2 -1 5
F -2 -3 -3 -3 -2 -3 -3 -3 -1 0 0 -3 0 6
P -1 -2 -2 -1 -3 -1 -1 -2 -2 -3 -3 -1 -2 -4 7
S 1 -1 1 0 -1 0 0 0 -1 -2 -2 0 -1 -2 -1 4
T 0 -1 0 -1 -1 -1 -1 -2 -2 -1 -1 -1 -1 -2 -1 1 5
W -3 -3 -4 -4 -2 -2 -3 -2 -2 -3 -2 -3 -1 1 -4 -3 -2 1 1
Y -2 -2 -2 -3 -2 -1 -2 -3 2 -1 -1 -2 -1 3 -3 -2 -2 2 7
V 0 -3 -3 -3 -1 -2 -2 -3 -3 3 1 -2 1 -1 -2 -2 0 -3 -1 4

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino-or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a protein” includes a plurality of such proteins and reference to “the protein” includes reference to one or more proteins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows an overview of the manufacturing process for producing β-trypsin.

FIG. 2 shows an SDS-PAGE gel (coomassie blue stain) demonstrating that trypsinogen has been successfully expressed (lane 1), is present in the elution subsequent to cation exchange chromatography (CEX) (lane 2), and in the filtrate subsequent to renaturing and filtration (lane 3).

FIG. 3 shows an SDS-PAGE gel (coomassie blue stain) demonstrating that β-trypsin is present in the second peak during affinity chromatography purification, and thus can be separated from α-trypsin, which is present in the first peak.

FIG. 4 shows protein yield and purity provided at different steps of the method as claimed. Results of three independent runs are shown. A: Table demonstrating protein yields. B: SDS-PAGE gel (Coomassie blue stain) demonstrating increasing purity as the method proceeds, showing significantly reduced contaminants when performing AEX subsequent to CEX (compare lanes 5 and 7). EsmunoS=CEX; EsmunoQ=AEX.

FIG. 5 shows RP-HPLC profiles of trypsinogen demonstrating stability at 2-8° C. for up to 40 hours, and a lack of stability at higher temperatures (ambient/room temperature). A: RP-HPLC of trypsinogen when refolding at ambient temperature leads to aggregation and reduced yield of refolded trypsinogen. Refolding was performed as described in the Examples (at 0.1 mg/ml in refolding buffer having 20 mM Tris, 1.5 mM GSH, 1.5 mM GSSG, 0.75 M L-Arginine, pH 8). B: RP-HPLC profile of refolded, filtered trypsinogen in SH-02 buffer demonstrates stability at 2-8° C. for up to 40 hours.

FIG. 6A shows RP-HPLC profiles of renatured trypsinogen after refolding in different L-arginine concentrations (with a starting protein concentration of ˜0.1 mg/ml). B—RP-HPLC profiles of renatured trypsinogen after refolding with different (denatured) trypsinogen concentrations (at 2-8° C. in refolding buffer with 0.75 M L-Arg).

FIG. 7A shows a benzamidine affinity chromatography trace, demonstrating separation of peaks for alpha-trypsin (1^{st} peak) and beta-trypsin (2^{nd} peak). B—SDS-PAGE analysis demonstrates separation of alpha-and beta-trypsin by benzamidine affinity chromatography.

### EXAMPLES

### Materials and Methods

### Bacterial Strain

E. coli BL21 (DE3) was transformed with bovine trypsinogen expression vector (a pET-28b+ plasmid harbouring the sequence of SEQ ID NO: 1 under the control of an IPTG inducible promoter). For convenience, this strain will be referred to as “RCB” throughout the Examples.

### Buffers and Culture Media

### Inoculum Medium:

Inoculum (pre-culture) medium composition is outlined below. This medium comprises four solutions, which are prepared and sterilised by autoclaving (121° C., 1 atm, 20 mins) separately. Solutions were then mixed (under laminar flow) prior to use to provide the inoculum medium:

| Component | Volume | | Composition of | Amount of | | |
|---|---|---|---|---|---|---|
| solutions | per prep. | | component solution | reagent | | Notes |
| Solution N | 920 | mL | Potassium dihydrogen phosphate | 1.82 | g | pH adjustment to |
| | | | Di-sodium hydrogen phosphate | 17.0 | g | 7.6-7.8 required |
| | | | Ammonium sulphate | 3.00 | g | |
| | | | PW | to final volume | | |
| | | | | of 920 mL | | |
| Solution P | 40 | mL | Magnesium sulphate heptahydrate | 0.50 | g | use prewarmed |
| | | | PW | to final volume | | water for solution |
| | | | | of 40 mL | | preparation |
| Solution C | 40 | mL | D(+)-glucose monohydrate | 15.0 | g | |
| | | | PW | to final volume | | |
| | | | | of 40 mL | | |
| Solution M | 80 | μL | Ferric chloride hexahydrate | 3000 | mg | same stock of M |
| | | | Calcium chloride dihydrate | 405.0 | mg | solution is used as |
| | | | Zinc sulphate heptahydrate | 675.0 | mg | supplement of |
| | | | Manganese sulphate monohydrate | 150.0 | mg | production |
| | | | Copper sulphate pentahydrate | 300.0 | mg | fermenter media |
| | | | Boric acid | 69.0 | mg | and feed solution |
| | | | Cobalt (II) chloride hexahydrate | 114.0 | mg | 3.7% HCl is |
| | | | Sodium molybdate dihydrate | 30.0 | mg | prepared from 37% |

| | | |
|---|---|---|
| 3.7% HCl | to final volume | HCl stock solution |
| | of 100 mL | |

### Production Culture Medium:

Production culture medium (e.g. minimal fermenter medium) composition is outlined below. Prior to use, P1 and C1 were sterilised by autoclaving (at 121° C., 1 atm, 20 mins). To prepare the production culture medium 1100 mL of N1 solution was sterilised by autoclaving (at 121° C., 1 atm, 30 mins) in a fermenter. The production culture medium was pre-warmed to +37° C. prior to inoculation. The production medium was (solution M same as in table above):

| Component | Volume | | Composition of | Amount of | | |
|---|---|---|---|---|---|---|
| solutions | per prep. | | component solution | reagent | | Notes |
| Solution N1 | 1.1 | L | Potassium dihydrogen phosphate | 15.52 | g | pH adjustment to 5.60- |
| | | | Ammonium phosphate dibasic | 4.67 | g | 5.90 required before |
| | | | Citric acid monohydrate | 1.87 | g | sterilization |
| | | | PW | to final volume | | |
| | | | | of 1.1 L | | |
| Solution P1 | 10 | mL | Magnesium sulphate heptahydrate | 0.58 | g | use pre-warmed |
| | | | PW | to final volume | | water for solution |
| | | | | of 10 mL | | preparation |
| Solution C1 | 90 | mL | D(+)-glucose monohydrate | 35.0 | g | |

| | | | | |
|---|---|---|---|---|
| | | | PW | to final volume |
| | | | | of 90 mL |
| Solution M | 292 | μL | | |

### C2+P2+M Solution

An additional glucose, magnesium and trace elements feeding solution (referred to a C2+P2+M solution) was use during biosynthesis. Prior to use, prepared solutions were sterilised by autoclaving (at 121° C., 1 atm, 20 mins). Each of solution P2, C2 and M were then mixed (under laminar flow) prior to use. The “C2+P2+M” solution was (solution M same as in table above):

| Component | Volume | | Composition of | Amount of | | |
|---|---|---|---|---|---|---|
| solutions | per prep. | | component solution | reagent | | Notes |
| Solution P2 | 15 | mL | Magnesium sulphate | 10.37 | g | use pre- |
| | | | heptahydrate | | | warmed water |
| | | | PW | to final volume | | for solution |
| | | | | of 15 mL | | preparation |
| Solution C2 | 485 | mL | D(+)-glucose monohydrate | 350.0 | g | |

| | | | | |
|---|---|---|---|---|
| | | | PW | to final volume |
| | | | | of 485 mL |
| Solution M | 1.7 | mL | | |

### N2 Solution

N2 solution was sterilised prior to use by autoclaving (at 121° C., 1 atm, 20 mins). During production culture, the addition of N2 solution (outlined below) was performed at fixed times. Four additions of N2 solution were used, as follows (volumes are for a 1.2 L w/v fermentation process, and are increased proportionally for larger scaled fermentation processes): (1) 25 mL when OD600 nm is 60-70 O.U.; (2) 25 mL when OD600 nm is 120-140 O.U.; (3) 12.5 mL at 1.5 hours after induction (IPTG); (4) 12.5 mL at 2 hours after induction. N2 solution was:

| | Amount of | |
|---|---|---|
| Component | reagent | Notes |
| Potassium dihydrogen | 23.0 g | use pre-warmed water |
| phosphate | | for solution preparation |
| Ammonium phosphate dibasic | 27.0 g | |
| PW | to final volume | |
| | of 75 mL | |

### Buffers

The following table outlines the buffers employed during the various method steps of the invention. For convenience, the ‘buffer identifiers’ are referred to throughout the Examples.

| | |
|---|---|
| Buffer | |
| Identifier | Buffer Details |

Upstream process

| | |
|---|---|
| N1 | Potassium dihydrogen phosphate (15.52 g); Ammonium phosphate |
| | dibasic (4.67 g); Citric acid monohydrate (1.87 g); PW (to final volume |
| | of 1.1 L) |
| | pH adjusted to pH 5.6-6.9 prior to sterilisation |
| P1 | Magnesium sulphate heptahydrate (0.58 g); PW (to final volume of 10 ml) |
| | Pre-warmed to 37° C. prior to use |
| C1 | D (+) glucose monohydrate (35 g); PW (to final volume of 90 ml) |
| | Pre-warmed to 37° C. prior to use |
| M | Iron(II) chloride hexahydrate (3 g); |
| | Calcium chloride dehydrate (405 mg); Zinc sulfate heptahydrate |
| | (675 mg); Manganese (II) sulfate monohydrate (150 mg); Copper |
| | sulfate pentahydrate (300 mg); Boric acid (69 mg); Cobalt(II) chloride |
| | hexahydrate (114 mg); Sodium molybdate dehydrate (30 mg); 3.7% |
| | HCL solution (to final volume of 100 ml) |
| N2 | Potassium dihydrogen phosphate (23 g); Ammonium phosphate |
| | dibasic (27 g); Citric acid monohydrate; PW (to final volume of 75 mL) |
| P2 | Magnesium sulfate heptahydrate (23 g); PW (to final volume of 15 ml) |
| | Pre-warmed to 37° C. prior to use |
| C2 | D(+) glucose monohydrate (350 g); PW (to final volume of 485 ml) |
| | Pre-warmed to 37° C. prior to use |
| P3 | Magnesium sulfate heptahydrate, 2.8M |
| C3 | D(+) glucose monohydrate, 4M |
| N3 | Potassium dihydrogen phosphate, 2.2M; Ammonium phosphate |
| | dibasic, 2.7M |
| Antifoam | 20% antifoam 204 |
| I | Isopropyl-β-D-thiogalactopyranoside (IPTG) |
| PW | Purified Water |

Midstream Process

| | |
|---|---|
| SM-E01 | 100 mM Tris, 5.0 mM Titriplex III, 0.1% TritonX-100 |
| SM-F01 | 10 mM Tris, 1.5M NaCl, 1% TritonX-100, 5 mM EDTA solution SM-F01 |
| SM-F02 | 10 mM Tris, 5 mM EDTA |

Downstream Process

| | |
|---|---|
| S-01 | 1M NaOH |
| S-02 | 2M NaCl |
| S-03 | 0.1M Acetic acid |
| S-04 | 0.5M NaOH |
| S-05 | 0.1M NaCl |
| S-06 | 1M NaCl |
| S-07 | 1M Citric acid |
| S-08 | 0.1M NaOH |
| S-09 | 0.15M NaCl, 20 Ethanol |
| S-10 | 0.1M Phosphoric acid |
| S-11 | 5M HCl |
| S-12 | 5M CaCl2 |
| S-13 | 6M Guanidine. HCl |
| S-14 | 5M NaOH |
| S-F01 | 8M Urea, 20 mM Citric acid, 0.1M DTT, pH 5.0 |
| S-F02 | 8M Urea, 20 mM Citric acid, pH 5.0 |
| S-G01 | 8M Urea, 20 mM Citric acid, 0.1M NaCl, pH 5.0 |
| S-H01 | 0.75M L-Arginine, 20 mM Tris, 1.5 mM GSH (reduced glutathione), |
| | 1.5 mM GSSG (oxidized glutathione), pH 8.0 |
| S-H02 | 50 mM Tris, pH 7.4 |
| S-K01 | 50 mM Tris, 0.5M EDTA, pH 8.0 |
| S-K02 | 50 mM Tris, pH 7.4 |
| S-L01 | 30 mM sodium di-hydrogen phosphate, 15 mM sodium citrate, pH 7.4 |
| S-L02 | 30 mM Di-sodium hydrogen phosphate, 15 mM citric acid, pH 3.0 |
| S-M01 | 10 mM Citric acid, pH 3.0 |

### Example 1

### Manufacturing β-trypsin Using an E. coli Heterologous Expression System

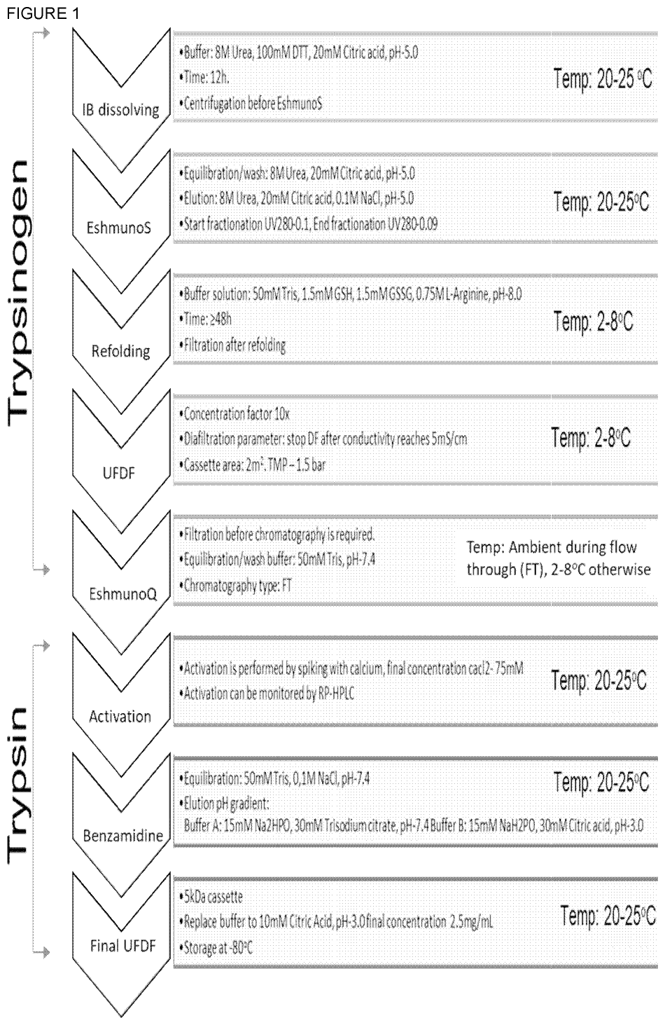

The β-trypsin was manufactured in accordance with the manufacturing process provided in FIG. 1. Further details on the manufacturing process are provided below.

### 1.1. Bacterial Culture and Cultivation Process

An inoculum culture was first prepared (using inoculum medium). Three 1000 mL shake flasks were provided, and 460 mL (+/−10 mL) of inoculum medium was added to each flask and heated to 30° C. A vial of RCB suspension (cryofrozen) was thawed (5-20 mins), and 1-1.5 mL of RCB was seeded into the inoculum medium in the flasks. The flasks were incubated for 17-18 hours at 30° C. (+/−2° C.), shaking at 300 (+/−50) RPM to provide the inoculum culture.

A production culture was then prepared. 10 mL of production medium was inoculated with inoculum culture, to provide an initial OD600 nm (of production culture) of 0.5 O.U. Fermentation conditions were as follows:
Temperature set point 37+/−2° C.
DO set point 20%
Air flow 0.3-7 L/min
Agitation (stirring) 300 rpm
Oxygen 0-7 L/min
pH set point 6.8+/−0.2 (adjusted by addition of NH₄OH)

During culturing, samples were taken for optical density and glucose concentration measuring. When the OD600 nm reached 20 O.U., feeding with glucose (P2+C2+M solution) was initiated. When the OD600 nm reached 60 O.U., the addition N2 solution began (further details provided under the header ‘N2 solution’ above). Expression of trypsinogen was induced by addition of IPTG (to a final concentration of 0.5 mM) when the OD600 nm of the production culture was ˜50. Induction was carried out for about 7 hours.

The production culture was harvested 7 hours after induction, by centrifugation at 8000 rpm for 20-30 mins (at 4° C.). The cell pellet was collected and stored at −20+/−5° C. until further use.

Three 10 L production runs (batches) were performed to monitor consistency of biomass yield and trypsinogen expression. Results from the three runs are outline below:

| | | | Trypsinogen expression |
|---|---|---|---|
| | Final | Biomass | level by SDS-PAGE, % |
| Batch No. | OD 600 nm | yield, g/L | from insoluble proteins |
| F24-BTPH-051- | 96 | 110.5 | 27 |
| 1803M-089 | | | |
| F24-BTPH-051- | 87 | 115.9 | 28 |
| 1803M-095 | | | |
| F24-BTPH-051- | 88 | 118.6 | 32 |
| 1803M-096 | | | |

### 1.2. Inclusion Body (IB) Extraction and Washing

Pellets were resuspended in SM-E01 buffer (at a temperature of 2-8° C.) at a ratio of 1:4. Resuspension was performed for 20-40 mins. Cell disruption (to extract IBs) was performed using a GEA Panda homogeniser at a pressure of 650-750 bar, for 2 cycles at 2-8° C. The pH was then adjusted to 7.4-7.6 when the temperature of the lysate reached 5-15° C. The lysate (including the IBs) was then centrifuged at 8000 rpm (Avanti J-26XPI) for 50-55 min (temperature: 2-8° C.).

First IB washing: Pellets (of lysate) were then washed with SM-F01 buffer at a dilution ratio of 1:15 and resuspended in the SM-F01 buffer (by via the homogeniser for 15-25 mins). The resuspended material was then centrifuged at 8000 rpm (Avanti J-26XPI) for 25-35 min (temperature: 2-8° C.).

Second and third IB washing: pellets were then washed with SM-F02 buffer at a dilution ratio of 1:15 and resuspended in the SM-F02 buffer (by via the homogeniser for 15-25 mins). The resuspended material was then centrifuged at 8000 rpm (Avanti J-26XPI) for 15-25 min (temperature: 2-8° C.). This was repeated for the third IB washing. IBs were then in stored (in bags) at 20+/−5° C. until further use.

### 1.3. IB Dissolving

IBs were resuspended in S-F01 buffer at a ratio of 1:19 (e.g. 1 g of IB per 19 grams of buffer). Resuspension was via the homogeniser for 12-15 hours, mixing at 250-400 rpm until completely dissolved. The solubilised material was then centrifuged at 800 rpm (Avanti J-26XPI-JLA-8.1000 rotor) for 50-60 mins (temperature 2-8° C.). The supernatant was then retained for further use and filtered with a 0.2 μm filter (Opticap XL300 or greater, with a filtration speed of 50-100 LHM and filter throughput of ˜4 L/m²).

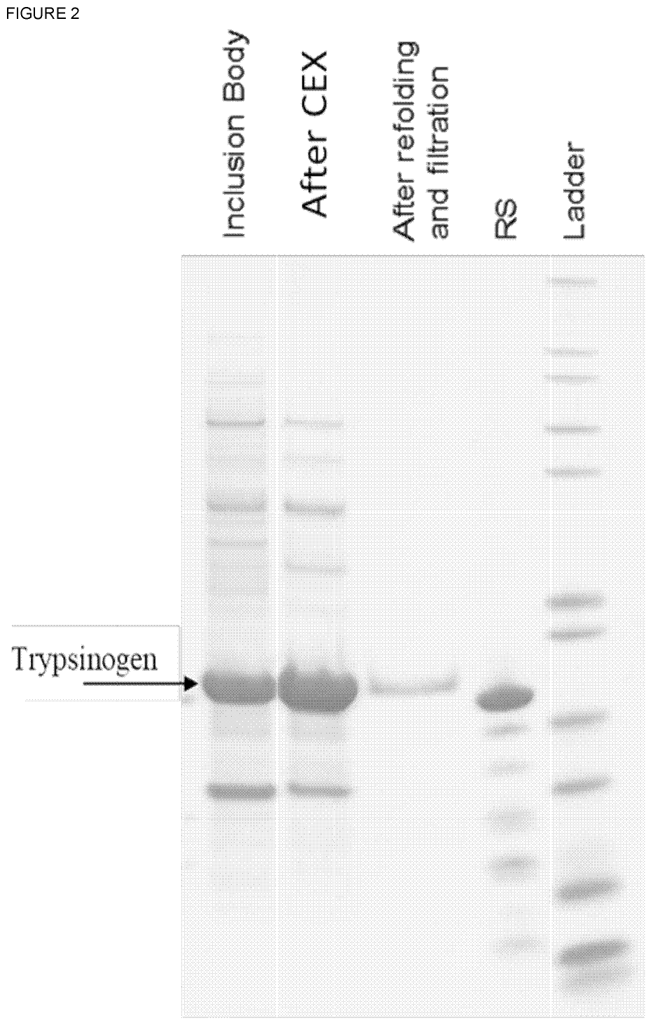

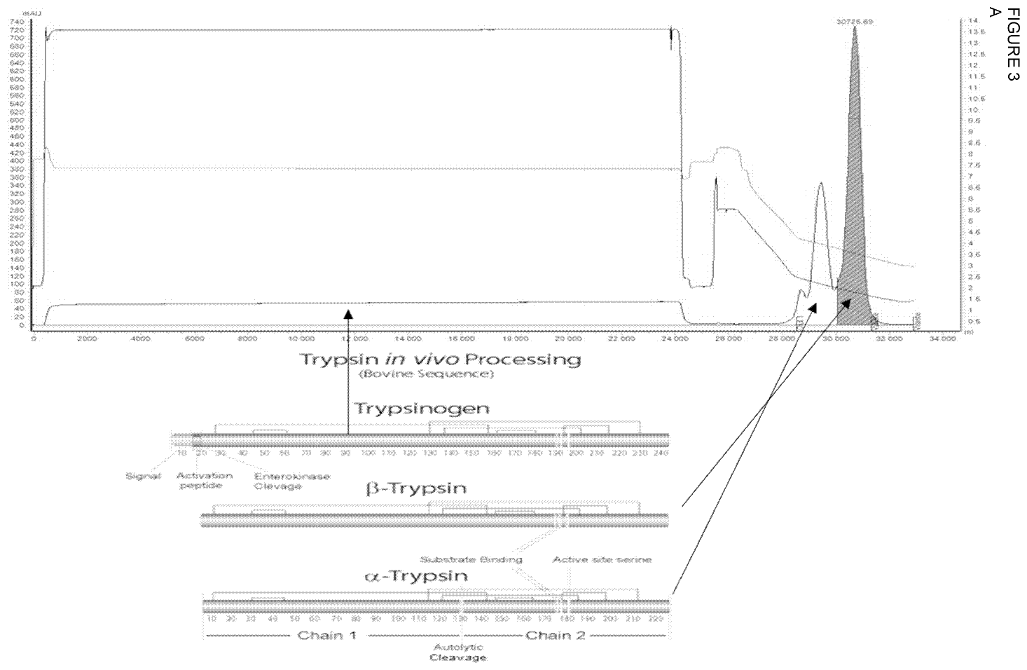

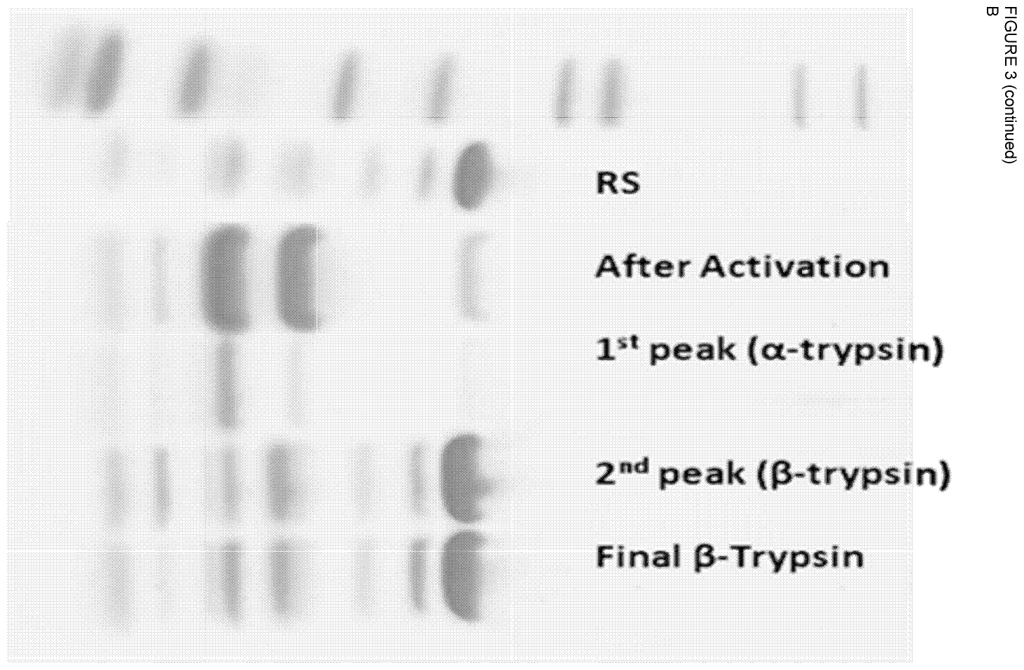

An aliquot of the supernatant was run on an SDS-PAGE gel to confirm the presence of trypsinogen (see FIG. 2).

### 1.4. Purifying Denatured Trypsinogen

Denatured trypsinogen was purified by cation exchange chromatography, using columns having EshmunoS resin (Merck Millipore). This allowed removal of host cell proteins, host cell DNA, bacterial endotoxins and protein related impurities, as well as concentration of trypsinogen before refolding.

The column had a volume of 1492-1649 mL, and a 20 cm bed height. The column was packed to achieve to following parameters: HETP (cm) of <=0.06, and A_{S} of 0.6-1.8.

The column was equilibrated with S-F02 buffer—flow rate, cm/h: target=75, range=60-80; column volume, CV: target=7, range=6-8.

Cation exchange chromatography (run in bind and elute mode) was performed according to the following parameters:

| Method | | | Flow | Flow rate, cm/h | | Solution volume, CV | |
|---|---|---|---|---|---|---|---|
| step | Solution | Inlets | Direction | Required | Fixed | Required | Fixed |
| Load | Filtered | S1 | Down | 70-80 | 75 | NA | |
| | supernatant | | | | | | |
| Wash | S-F02 | A1 | Down | 70-80 | 75 | 4.9-5.1 | 5 |
| Elution | S-G01 | A2 | Down | 70-80 | 75 | 6.9-7.1 | 7 |

Fractionation began at UV280-0.1 and was ended at UV280-0.09.

Eluted material was diluted to a volume of 10 L with S-G01 buffer. An aliquot of eluted material was run on an SDS-PAGE gel to confirm the presence of trypsinogen (see FIG. 2).

A more detailed overview of the cationic exchange chromatography conditions is as follows:

| | Flow rate, cm/h | | Column volume, CV | |
|---|---|---|---|---|
| Sub-process | Target | Range | Target | Range |
| Equilibration (8M Urea, 50 mM | 75 | 60-80 | 7 | 6-8 |
| Citric acid, pH-5.0) | | | | |
| Load by Lowry | 75 | 60-80 | <70 g/L | NA |
| Wash (8M Urea, 50 mM Citric | 75 | 60-80 | 5 | 4.5-5.5 |
| acid, pH-5.0) | | | | |
| Elution (start collection when mAu > 100 | 75 | 60-80 | 4 | 3.5-4.5 |
| end when mAu < 90 on 2 mm UV cell length) | | | | |
| (8M Urea, 50 mM Citric acid, 0.1M NaCl | | | | |
| pH-5.0) | | | | |
| Regeneration with 1M NaOH | 75 | 60-80 | 3 | 2.5-3.5 |
| Regeneration with WFI | 75 | 60-80 | 3 | 2.5-3.5 |
| Regeneration with 1M NaOH | 75 | 60-80 | 3 | 2.5-3.5 |
| Regeneration with WFI | 75 | 60-80 | 3 | 2.5-3.5 |
| Regeneration with 0.5M Acetic acid | 75 | 60-80 | 3 | 2.5-3.5 |
| Regeneration with WFI | 75 | 60-80 | 6 | 5.5-6.5 |
| Storage in 20% Alcohol | 75 | 60-80 | 3 | 2.5-3.5 |

### 1.5. Refolding Trypsinogen and Filtration

The elution material was cooled to 2-8° C. (and refolding was conducted at this temperature).

Refolding was performed by addition of S-H01 buffer to the elution material, to provide 0.1 mg/ml protein in the buffer. The solution was mixed at 200 rpm and incubated for 48-168 hours at 2-8° C. The solution was subsequently filtered using an OpticapXL150 0.5/0.2 μm filter.

Advantageously, use of L-arginine as an aggregation inhibitor in S-H01 did not cause filter clogging in step 1.6 which occurred when PEG was employed as an aggregation inhibitor.

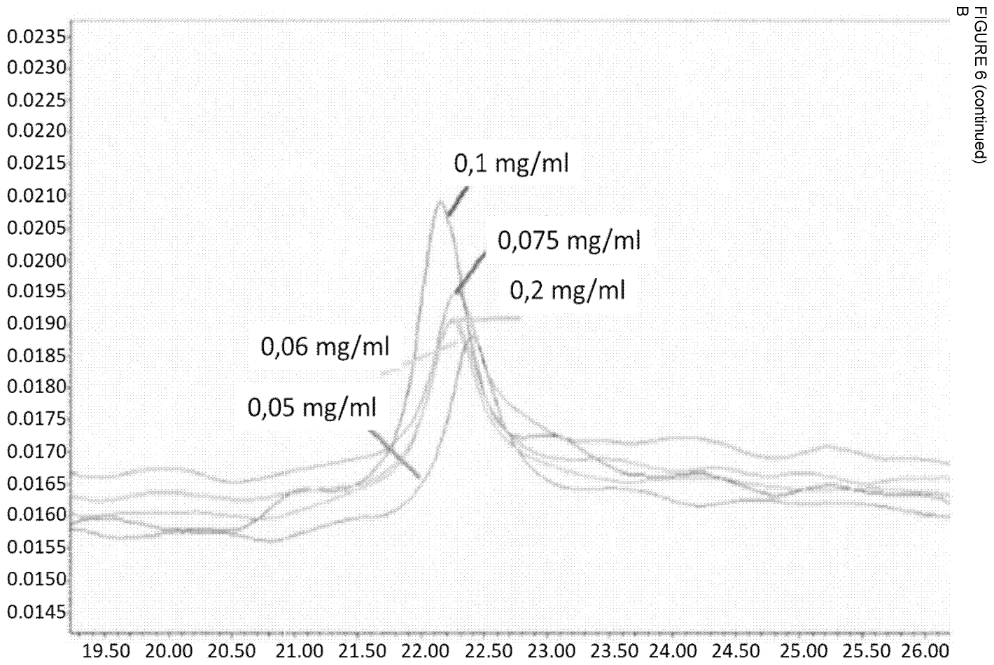

Varying concentrations of L-arginine for S-H01 buffer were tested, and RP-HPLC analysis (where a higher peak is indicative of higher refolding efficiency) revealed particularly advantageous L-arginine concentrations. In more detail, refolding of trypsinogen (at a protein concentration of 0.1 mg/ml) was tested in the presence of 0.5-0.75 M (at 0.05 M increments), revealing 0.75 M as the most advantageous concentration for refolding (see FIG. 6A).

Furthermore, a protein concentration of 0.1 mg/ml during refolding yielded greater yields of renatured trypsinogen versus higher (e.g. 0.2 mg/ml) or lower (e.g. 0.075 mg/ml), see Figure B.

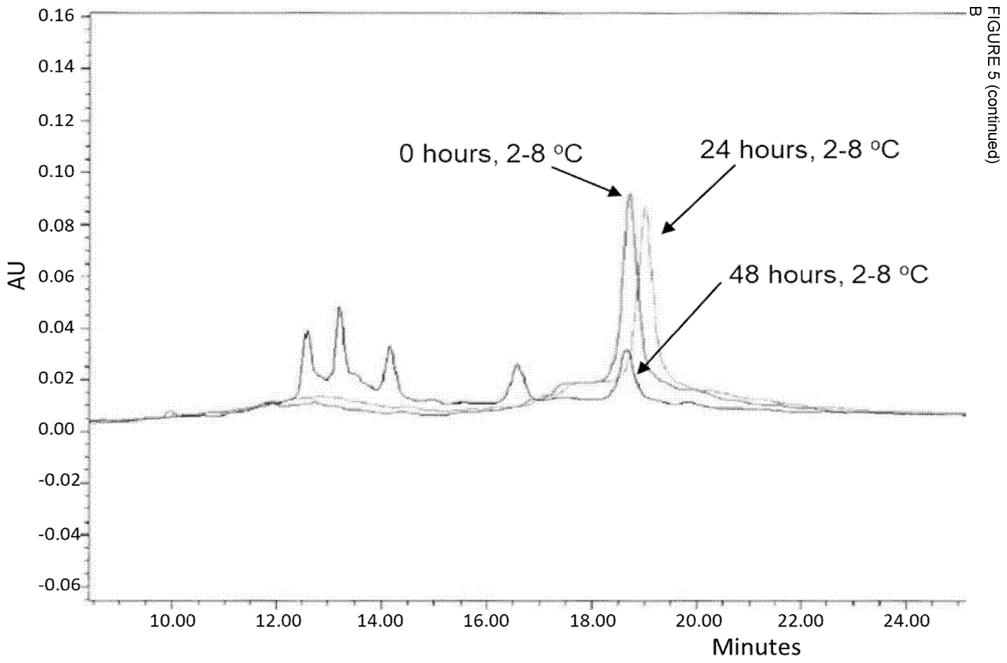

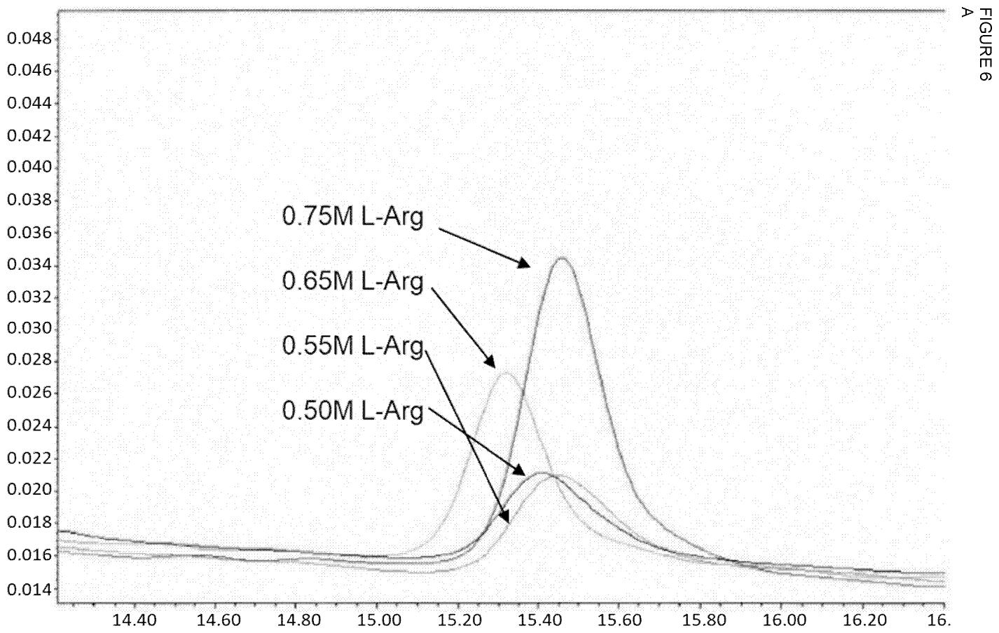

The inventors found that the trypsinogen remains stable at low temperature (e.g. 2-8° C.) for long periods of time (>=36 h, see FIG. 5A), allowing refolding to be conducted to completion.

The operational parameters for refolding are outlined below:

| | | |
|---|---|---|
| Parameter | Current Lab-scale | GMP (expectations) |

| | |
|---|---|
| Refolding buffer | 20 mM Tris, 1.5 mM GSH, |
| | 1.5 mM GSSG, 0.75M L-Arg, pH-8.0 |

| | | |
|---|---|---|
| Refolding volume, L | 10 | 200 |
| amount of protein after | <0.9 | <18 |
| EshmunoS, g | | |

### 1.6. Filtration Before UF/DF

Renatured trypsinogen was filtered through and Opticap XL150 0.5/0.2 μm at the maximum available speed in lab-scale which is 400LHM. The final operational parameters were as follows:

| Parameter | Current Lab-scale | GMP (1 gram scale) |
|---|---|---|
| Filter | Opticap XL150 | Opticap XL3 |
| | or bigger | or bigger |
| Filtration area, m2 | 0.015 | 0.13 |
| Filtration speed LHM | 200-400 | 200-400 |
| Filter Throughput (L/m2) | ~2000 | ~1540 |
| DSP comments | No clogging | No clogging |
| Duration, min | ~100 | ~200 min |

A chase buffer (50 mM Tris, pH 7.4) was used to flush any remaining refolded protein out of the filter, with the following operational parameters:

| Parameter | Current Lab-scale | GMP (1 gram scale) |
|---|---|---|
| Filter washing with | 1 | 1 |
| buffer, L/m2 | | |
| Filtration, bar | <2 | <2 |
| Filter wash, L/m2 | 5 | 5 |

### 1.7. Ultrafiltration/Diafiltration (Tangential Flow Filtration, TFF)

Tangential flow filtration was used both for volume reduction, as well as dialysis into a buffer (lacking L-Arg) suitable for the activation steps. This was conducted prior to anion exchange chromatography (AEX), such that the flow through (from the AEX column) was in a buffer ready for the activation step.

By using selective membrane sizes in tangential flow filtration, this filter setup can be used to perform protein concentration (ultrafiltration) and dialysis (diafiltration), typically short-handed as UF/DF. A molecular weight cut-off (MWCO) of 10 kDa (2-3 time lower than the target protein) was used. This size retains the target protein, but allows highly viscous L-arginine buffer to pass through. The ‘Millipore Pellicon 2, A-screen filter’ (MWCO 10 kDa) was used (no protein was observed in permeate).

The UF/DF allowed the protein volume to be reduced by 10×, as well as buffer exchange (diafiltration).

Thus, the solution (of renatured trypsinogen) was filtered with a filter (Millipore Pellicon 2) having a molecular weight cut-off of 10 kDa. The solution was concentrated by ten times. The operational conditions are outlined below:

| Parameter | Target | Range |
|---|---|---|
| Concentration factor | 10 | 9-11 |
| Retentate cross flow | 3.6 | 3-7 |
| (manufacture recommends), | | |
| L/min/m2 | | |
| Transmembrane Pressure | 1.5 bar | 1.4-1.6 bar |
| (TMP) | | |
| High alarm feed pressure | 4 Bar | — |

Buffer exchange (into S-H02 buffer) was then achieved using a diafiltration mode in which S-H02 buffer is added to the retentate system at the same rate which the current buffer (S-H01) is removed through the permeate. A diavolume (DV) is when the retenate volume of buffer has been exchanged. The diafiltration process ends when permeate conductivity and pH reaches the target value. The operational conditions are outlined below:

| Parameter | Target | Range |
|---|---|---|
| DV | 3.5x | >3.0 |
| Final pH | 7.5 | 7.4-7.6 |
| Final Conductivity | 6.5 | 5.0-8.0 |
| Retentate cross flow | 3-7 | 3-7 |
| (manufacture recommends), | | |
| L/min/m2 | | |
| Transmembrane Pressure | 1.5 | 1.4-1.6 |
| (TMP), bar | | |
| High alarm feed pressure, | 4 | — |
| bar | | |

An aliquot of the resulting TFF retentate was run on an SDS-PAGE gel to confirm the presence of trypsinogen (see FIG. 2).

Three batches were run (according to steps 1.1-1.6) to monitor consistency of biomass, IB and protein production. The results of three batches are outlined below:

| | | | Target protein | |
|---|---|---|---|---|
| | Biomass | | concentration, | |
| Batch | weight, g | Total IB, g | mg/g | Total target protein, g |
| P23-BTPH- | 1291 | 152 | 47 | 7.14 |
| 051-1701M- | | | | |
| 002 | | | | |
| P23-BTPH- | 1134 | 160 | 97 | 15.5 |
| 051-1701M- | | | | |
| 003 | | | | |
| P23-BTPH- | 1317 | 156 | 103 | 16.1 |
| 051-1701M- | | | | |
| 004 | | | | |

### 1.8. Purifying Renatured Trypsinogen

Renatured trypsinogen was purified by anion exchange chromatography (run in flow through mode), using columns having Eshmuno Q resin (Merck Millipore). Advantageously, this allowed further removal of bacterial endotoxins and host cell DNA, due to the net charge difference between trypsinogen and these impurities (such that the impurities are efficiently retained, thus removed from trypsinogen present in the flow-through).

The column had a volume of 177-216 mL, and a 10 cm bed height. The column was packed to achieve to following parameters: HETP (cm) of <=0.06, and A_{S} of 0.6-1.8.

The column was equilibrated with S-H02 buffer—flow rate, cm/h: target=150 cm/h, range=100-160 cm/h; column volume, CV: target=7, range=6-8. Thus, the residence time on the column is ˜4 mins. Flow through (comprising purified renatured trypsinogen) is collected immediately.

Anion exchange chromatography (run in flow through mode) was performed according to the following parameters:

| Method | | | Flow | Flow rate, cm/h | | Solution volume, CV | |
|---|---|---|---|---|---|---|---|
| step | Solution | Inlets | Direction | Required | Fixed | Required | Fixed |
| Load | TFF Retentate | S1 | Down | 145-155 | 150 | NA | |
| Wash | S-H02 | A1 | Down | 145-155 | 150 | 4-5 | Wash |

A more detailed overview of the anion exchange chromatography operational conditions is as follows (CV=column volume, e.g. 177-216 mL):

| | Flow rate, cm/h | | Column volume, CV | |
|---|---|---|---|---|
| Subprocess | Target | Range | Target | Range |
| 50 mM Tris, pH-7.4 | 150 | 100-160 | 7 | 6-8 |
| Load | 150 | 100-160 | N/A | N/A |
| NMT 100 g protein/L resin | | | | |
| Start collection immediately | | | | |
| when loading start | | | | |
| 50 mM Tris, pH-7.4 | 150 | 100-160 | 2 | 1.9-2.1 |
| End collection after 1.5CV | | | | |
| Regeneration with 1M sodium hydroxide | 60 | 30-70 | 3 | 2.5-3.5 |
| WFI | 150 | 60-160 | 3 | 2.5-3.5 |
| Regeneration with 2M Sodium hydroxide | 150 | 60-160 | 3 | 2.5-3.5 |
| WFI | 150 | 60-160 | 3 | 2.5-3.5 |
| Regeneration with 0.1M phosphoric acid | 150 | 60-160 | 3 | 2.5-3.5 |
| WFI | 150 | 60-160 | 6 | 5.5-6.5 |
| Storage in 20% ethanol | 60 | 30-70 | 3 | 2.5-3.5 |

The flow through material (comprising renatured trypsinogen) was collected.

### 1.9. Activation (Cleavage of Trypsinogen to Provide Trypsin) and Filtering

Activation was begun within 60 minutes of collecting the flow through material. Advantageously, by working within this timeframe, the inventors have found that aggregation of the trypsinogen protein (subsequent to anion exchange chromatography) can be mitigated (see Example 3).

The flow through material was at 20-25° C. prior to commencing activation. S-12 solution was added to the flow through material (to provide a final calcium concentration of 75 mM) and incubated for 20-24 hours at 20-25° C. Activation was quenched by addition of S-K01 buffer (final concentration 80 mM), and adjustment of pH to 7.2-7.6 with S-01 buffer.

The solution was then filtered through an Opticap XL150 0.5/0.2 μm filter. The operational parameters were as follows:

| Parameter | Current Lab-scale | GMP (1 gram scale) |
|---|---|---|
| Filter | Opticap XL150 or bigger | Opticap XL3 or bigger |
| Filtration area, m² | 0.015 | 0.13 |
| Filtration speed LHM | 200-400 | 200-400 |
| Filter Throughput (L/m²) | ~2000 | ~1540 |
| DSP Comments | No clogging | No clogging |

A chase buffer (S-H02) was used to flush any remaining refolded trypsinogen out of the filters. The operational parameters were as follows:

| Parameter | Current Lab-scale | GMP (1 gram scale) |
|---|---|---|
| Filter washing with buffer, | 1 | 1 |
| L/m² | | |
| Filtration, bar | <2 | <2 |
| Flush, L/m² | 5-8 | 5-8 |

### 1.10. Affinity Chromatography (Benzamidine)

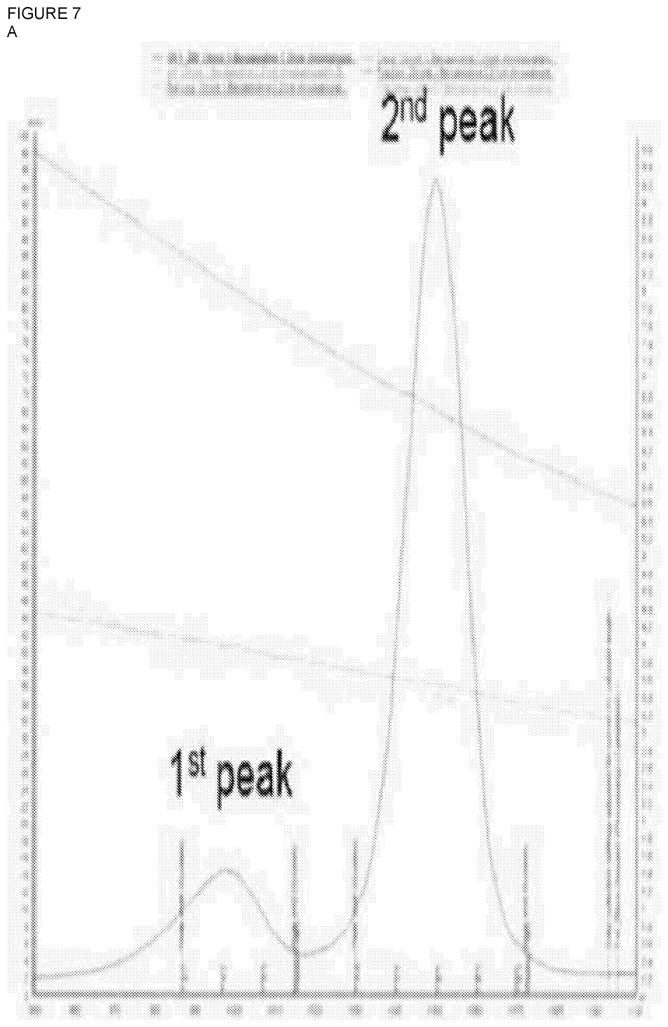

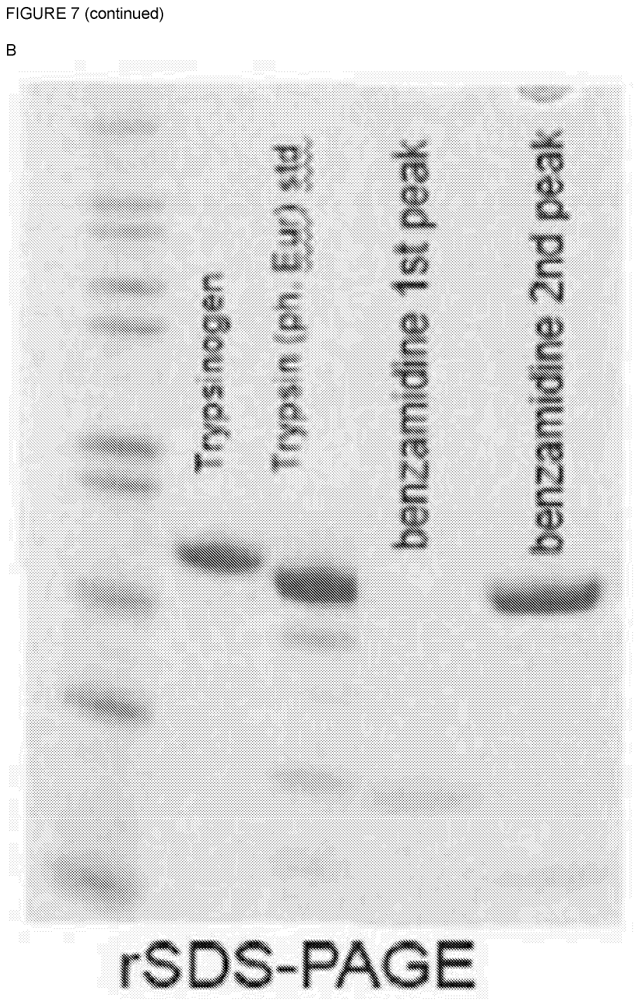

Activated trypsin was purified (e.g. polished) by affinity chromatography, using columns having Benzamidine Sepharose FF (Merck Millipore). This allowed further removal of bacterial endotoxins and host cell proteins, as well as trypsinogen which was not activated and α-trypsin. P-aminobenzamidine, a synthetic inhibitor of trypsin and trypsin like serine proteases, is covalently bound to a long spacer arm attached to Sepharose 4 fast flow. The benzamidine resin is used for separation of trypsin forms: alpha and beta. This step allowed separation of both forms (see FIG. 7).

The column had a volume of 177-216 mL, and a 10 cm bed height. The column was packed to achieve to following parameters: HETP (cm) of <=0.06, and A_{S} of 0.6-1.8.

The column was equilibrated with S-H02 buffer—flow rate, cm/h: target=150, range=100-160; column volume, CV: target=5, range=4.5-5.5.

Affinity chromatography was performed according to the following parameters:

| Method | | | Flow | Flow rate, cm/h | | Solution volume, CV | |
|---|---|---|---|---|---|---|---|
| step | Solution | Inlets | Direction | Required | Fixed | Required | Fixed |
| Load | Activated | S1 | Down | 145-155 | 150 | NA | |
| | material | | | | | | |
| Wash1 | S-K02 | A1 | Down | 145-155 | 150 | 2.5-3.5 | 3 |
| Wash2 | S-L01 | A2 | Down | 145-155 | 150 | 1.5-2.5 | 2 |
| Elution | S-L01 to S-L02 | A2/B2 | Down | 145-155 | 150 | 4.9-5.1 | 5 |
| | (0% −> 70%) | | | | | | |
| | S-L01 to S-L02 | A2/B2 | Down | 145-155 | 150 | 9.9-10.1 | 10 |
| | (70 −> 100%) | | | | | | |
| | S-L02 | B2 | Down | 145-155 | 150 | 1.9-2.1 | 2 |

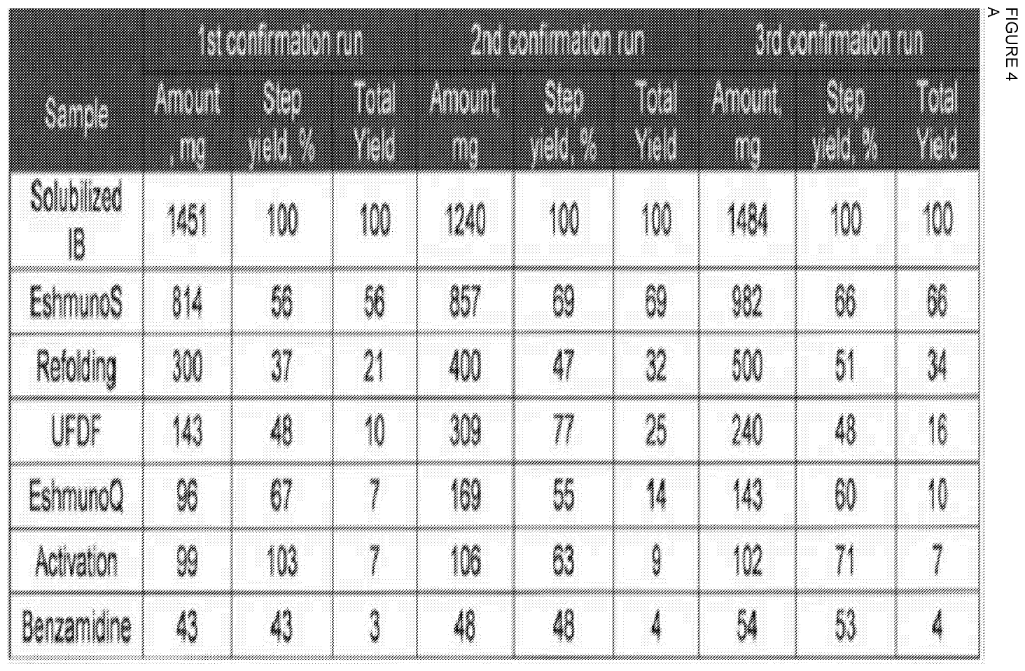

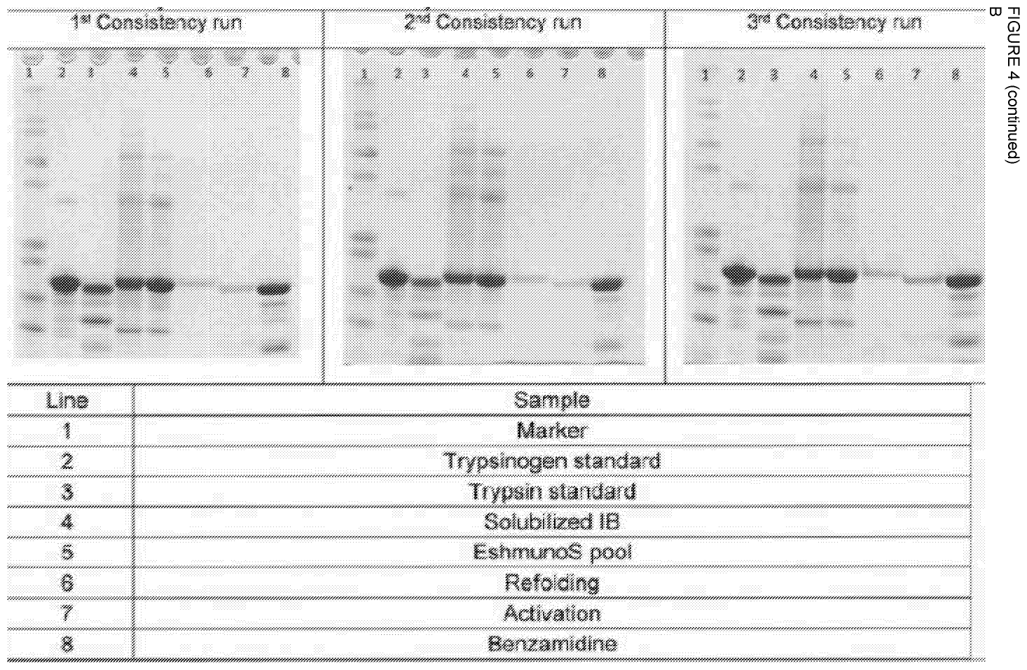

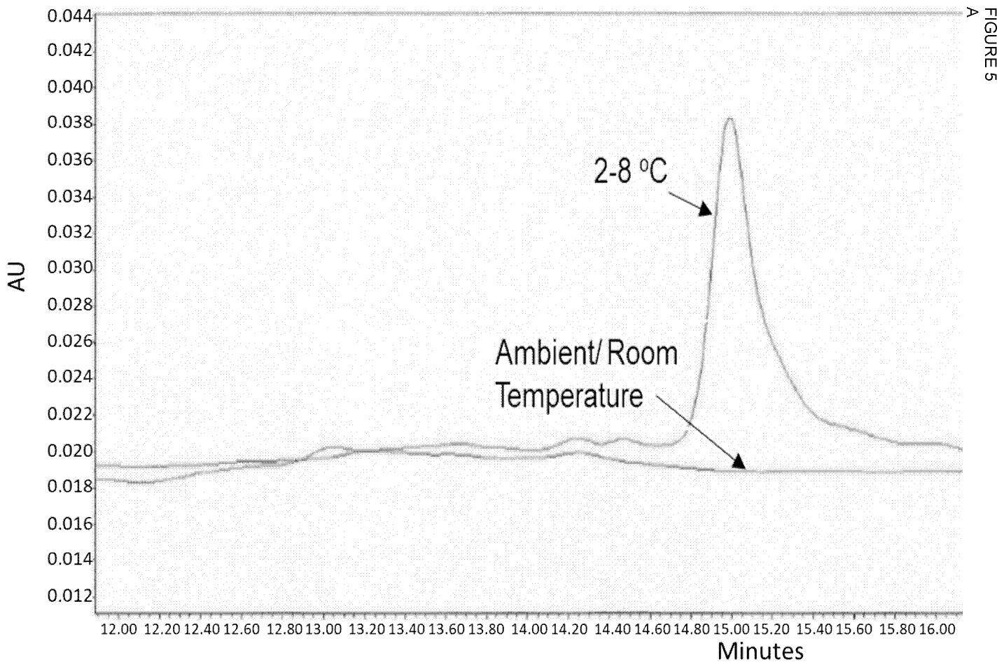

Elution was collected from 40 mAu to 30 mAu, and only second peak fractions were pooled (which contained β-trypsin). An aliquot of the first and second peaks were run on an SDS-PAGE gel to confirm the presence of β-trypsin in the second peak fraction (see FIG. 3).

A detailed overview of the benzamidine chromatography conditions is as follows:

| | Flow rate, cm/h | | Solution volume, CV | |
|---|---|---|---|---|
| Sub-process | Required | Fixed | Required | Fixed |
| Equilibration | 150 | 100- | 5 | 4.5- |
| (50 mM Tris, pH-7.4 at ambient conditions) | | 160 | | 5.5 |
| Load | 150 | 100- | N/A | N/A |
| NMT 4 g protein/L resin | | 160 | | |
| Wash 1 | 150 | 100- | 4 | 3.5- |
| (50 mM Tris, pH-7.4 at ambient conditions) | | 160 | | 4.5 |
| Wash 2 | 150 | 100- | 3 | 2.5- |
| (30 mM Na2HPO4, 15 mM sodium citrate, pH- | | 160 | | 3.5 |
| 7.4) | | | | |
| Gradient elution 1 | 150 | 100- | 5 | 4.5- |
| A buffer: 30 mM Na2HPO4, 15 mM sodium | | 160 | | 5.5 |
| citrate, pH-7.4 | | | | |
| B buffer: 30 mM Na2HPO4, 15 mM Citric acid, | | | | |
| pH-3.0 | | | | |
| 0% −> 70% (B buffer) | | | | |
| Gradient elution 2 | 150 | 100- | 10 | 9.5- |
| A buffer: 30 mM Na2HPO4, 15 mM sodium | | 160 | | 10.5 |
| citrate, pH-7.4 | | | | |
| B buffer: 30 mM Na2HPO4, 15 mM Citric acid, | | | | |
| pH-3.0 | | | | |
| 70% −> 100% B buffer | | | | |
| Elution 3 | 150 | 100- | 2 | 1.5- |
| 30 mM Na2HPO4, 15 mM Citric acid, pH-3.0 | | 160 | | 2.5 |
| Sanitization | 150 | 100- | 2 | 1.5- |
| (6M Guanidine) | | 160 | | 2.5 |
| WFI | 150 | 100- | 8 | 7.5- |
| | | 160 | | 8.5 |
| Storage | 60 | 100- | 3 | 2.5- |
| (20% Ethanol) | | 160 | | 3.5 |

### 1.11. Final Ultrafiltration/Diafiltration (Tangential Flow Filtration, TFF)

The elution (from affinity chromatography) was filtered with filter (Millipore Pellicon 2 A type cassette) having a molecular weight cut-off of 5 kDa (˜5 times smaller than the protein, to ensure the protein was retained while allowing buffer species to be removed). The elution was concentrated to provide a concentration of protein of ˜2 mg/ml. The operational conditions are outlined below:

| Parameter | Target | Range |
|---|---|---|
| Protein concentration, mg/mL | 2 | 2 |
| Retentate cross flow | 3-7 | 3-7 |
| (manufacture recommends), | | |
| L/min/m2 | | |
| Transmembrane Pressure | 1.0 | 0.8-1.4 |
| (TMP), bar | | |
| High alarm feed pressure, bar | 4 | — |

Buffer exchange (into S-M01 buffer) was then achieved using a diafiltration mode in which the buffer is added to the retentate system at the same rate which the current buffer (S-L02) is removed through the permeate. A diavolume (DV) is when the retenate volume of buffer has been exchanged. After 5 DVs, 99.9% of the old buffer was removed. The diafiltration process ends when permeate conductivity and pH reaches the target value. The operational conditions are outlined below:

| Parameter | Target | Range |
|---|---|---|
| DF volume | 7x | ≥7x |
| Final pH | 3.0 | 2.8-3.2 |
| Retentate cross flow | 4-7 | 4-7 |
| (manufacture recommends), | | |
| L/min/m² | | |
| Transmembrane Pressure | 1.0 | 0.8-1.4 |
| (TMP), bar | | |
| High alarm feed pressure, Bar | 4 | — |

### 1.12. Final Concentration and Storage

The final ultrafiltration step brings the protein concentration to 2.5 mg/ml (via diafiltration using a 0.2 μm filter as above). The operation parameters were as follows:

| Parameter | Target | Range |
|---|---|---|
| Target protein concentration, | 2.5 | 3-4 |
| mg/mL | | |
| Retentate cross flow | 4-7 L/min/m2 | 4-7 L/min/m2 |
| (manufacture recommends) | | |
| Transmembrane Pressure | 1.0 bar | 0.8-1.4 bar |
| (TMP) | | |
| High alarm feed pressure | 4 Bar | — |

The product was then dispensed into 20 mL aliquots in 30 mL PETG bottles, and stored at −80+/−10° C.

The purity of β-trypsin in the final filtrate was found to be >90% (measured via SDS-PAGE).

The product was lyophilised to a Drug Product using the following cycle parameters:

| | |
|---|---|
| Shelf Temperature Range | −45° C. to +30° C. |
| Shelf Freezing Rate | 0.5° C./min |
| Shelf Heating Rate | 1-5° C./min |
| Chamber Pressure range | 50 mTorr to 135 mTorr |

### Example 2

### Suppression of Trypsinogen Instability Post-Anion Exchange Chromatography

Renatured trypsinogen was provided according to steps 1-1.8 of Example 1.

Before conducting step 1.9 (activation via cleavage of trypsinogen to provide trypsin), the renatured trypsinogen (the output of step 1.8) was held for different time points at room temperature (e.g. ˜20° C.), namely timepoints of ≤60 minutes or >60 minutes, and protein stability was subsequently assessed via visual inspection (where cloudiness in the protein preparation indicated lack of stability, and no cloudiness indicated stability). Advantageously, a max hold time of 60 mins was shown to prevent the emergence of cloudiness, demonstrating the ability to suppress trypsinogen instability post-AEX by conducting step 1.9 within a maximum of 60 mins.

| | Cloudiness (e.g. |
|---|---|
| Time held post-AEX | precipitation) observed |
| ≤60 minutes | No |
| >60 minutes | Yes |

### Example 3

### Measuring the Cleavage Activity of β-trypsin Produced by a Method of the Invention

Trypsin activity of the purified β-trypsin using Nα-Benzoyl-L-arginine ethyl ester (BAEE) as the substrate. This procedure is a continuous spectrophotometric rate determination (A₂₅₃, Light path=1 cm) based on the following reaction:

BAEE+H₂O (in the presence of Trypsin)>Nα-Benzoyl-L-arginine+ethanol

where:

BAEE-N_{α}-Benzoyl-L-arginine ethyl ester

Unit Definition—One BAEE unit of trypsin activity will produce a ΔA₂₅₃ of 0.001 per minute with BAEE as substrate at pH 7.6 at 25° C. in a reaction volume of 3.20 ml.

### Reagents and Equipment:

Sodium phosphate, monobasic (e.g. Sigma Catalog No. S0751), Nα-Benzoyl-L-arginine ethyl ester (BAEE, e.g. Sigma Catalog No. B4500), 1 M NaOH solution, 1 M hydrochloric acid.

### Preparation Instructions

Ultrapure water (≥18 MΩ×cm resistivity at 25° C.) is used for the preparation of reagents.
Buffer (67 mM Sodium Phosphate Buffer, pH 7.6 at 25° C.)—Prepare a 8.04 mg/ml solution using sodium phosphate, monobasic in ultrapure water. Adjust to pH 7.6 at 25° C. with 1 M NaOH solution.
Substrate Solution (0.25 mM Nα-Benzoyl-L-arginine ethyl ester)—Prepare a 0.086 mg/ml solution using Nα-Benzoyl-L-arginine ethyl ester (BAEE, e.g. Sigma Catalog No. B4500) in Buffer.
HCl Solution (1 mM Hydrochloric Acid)—Prepare a 1,000-fold dilution of 1 M Hydrochloric acid solution in ultrapure water.
Enzyme Solution (Trypsin)—Immediately before use, the trypsin composition (both the test sample and control reference sample) is diluted to 1 mg (of polypeptide)/ml in cold (2□8° C.) HCl Solution.

### Procedure:

In a 3.20 ml reaction mix, the final concentrations are 70 mM (e.g. 62.8 mM) sodium phosphate, 0.23 mM Nα-Benzoyl-L-arginine ethyl ester, 0.031-0.063 mM hydrochloric acid, 42.5-115.0 units of trypsin.
1. Pipette the following reagents into suitable quartz cuvettes:

| Reagent | Blank (ml) | Test (ml) |
|---|---|---|
| Substrate Solution | 3.00 | 3.00 |
| HCl Solution | 0.200 | 0.125 |

2. Mix by inversion and equilibrate to 25° C. using a suitably thermostatted spectrophotometer. Then add:

| Reagent | Blank (ml) | Test (ml) |
|---|---|---|
| Enzyme Solution | — | 0.075 |

3. Immediately mix by inversion and record the increase in A253 for 5 minutes. Using a 1 minute time period and 4 data points, obtain the ΔA₂₅₃/minute using the maximum linear rate for both the Blank and Tests.

### Calculations:

$$Activity BAEE U / ml = \frac{( \frac{{A}_{2} - {A}_{1}}{t} ) \times D F}{{A}_{253 / min} \times V}$$

A₁ is the A₂₅₃ immediately after preparing the admixture;
A₂ is the A₂₅₃ of the admixture after 4 minutes of incubation,
t is the duration of the incubation;
DF is the dilution factor;
A_{253/min} is 0.001; and
V is the volume of the β-trypsin composition in the admixture, 0.075 mL
   or after obtaining the ΔA₂₅₃/minute using the maximum linear rate for both the Blank and Tests:

$$BAEE units / ml enzyme = \frac{( Δ {A}_{253} / minute Test - Δ {A}_{253} / minute Blank ) \times ( df )}{( 0.001 ) \times ( 0.075 )}$$

wherein: df is the dilution factor;
   0.001=the change in A₂₅₃/minute based on unit definition;
   0.075 ml=the volume of the β-trypsin composition Test.

Convert BAEE units/ml to USP units/mg by dividing the BAEE U/ml by the concentration of the β-trypsin composition (e.g. mg/ml of polypeptide) and subsequently dividing by three.

### Results:

Activity was compared directly with that of a reference standard (RS). RS=Trypsin BRP (Eur. Ph.) Reference Standard from EDQM Cat No. T2600000 batch 2 diluted to 1 mg/ml. The test sample (e.g. β-trypsin composition produced by a method of the invention) was also diluted to 1 mg/ml. The activity of β-trypsin produced by the present invention was 12,342 BAEE U/mg. The activity of RS was 8,233 BAEE U/mg. Thus, the activity of the claimed β-trypsin composition is approximately 50% greater than that of the RS.

Certain properties of the β-trypsin composition was assessed (sample 1=liquid trypsin release, sample 2=lyo trypsin release), see table below:

| Properties | Attribute/method | Sample 1 | Sample 2 |
|---|---|---|---|
| Potency | Activity assay | ≥3000 USP units/mg | ≥3000 USP units/mg |
| Purity, impurities | Truncated forms | SDS-PAGE band pattern | SDS-PAGE band pattern |
| and | (reduced SDS-PAGE) | consistent with reference | consistent with reference |
| contaminants: | | standard | standard |
| product-related | Bacterial endotoxins | ≤500 EU/mL | ≤500 EU/mL |
| | Microbial contamination | — | Microbial contamination |
| | (bioburden) | | TAMC: acceptance criterion |
| | | | 104 CFU/g (2.6.12). TYMC: |
| | | | acceptance criterion 102 |
| | | | CFU/g (2.6.12). Absence of |
| | | | Escherichia coli (2.6.13). |
| | | | Absence of Salmonella (2.6.13) |
| Quantity | pH | 2.8-3.2 | 2.8-3.2 |
| | Appearance | Clear colourless liquid | White/off white powder |
| | | essentially free from visible | |
| | | impurities | |
| General/other | ID (Eur. PH.) | — | Conforms to test |
| | Container/Closure | — | None of the vials contain any |
| | Integrity (CCI) | | trace of coloured solution |
| | Moisture | — | ≤1.5 |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

SEQUENCES

SEQ ID NO: 1 (Bovine trypsinogen)
CATATGTAGAAGGAGATATACCATGGTGGATGATGATGATAAAATTGTGGGCGGCTATACCT
GCGGCGCGAACACCGTGCCGTATCAGGTGAGCCTGAACAGCGGCTATCATTTTTGCGGCGGC
AGCCTGATTAACAGCCAGTGGGTGGTGAGCGCGGCGCATTGCTATAAAAGCGGCATTCAGGT
GCGCCTGGGCGAAGATAACATTAACGTGGTGGAAGGCAACGAACAGTTTATTAGCGCGAGC
AAAAGCATTGTGCATCCGAGCTATAACAGCAACACCCTGAACAACGATATTATGCTGATTAAA
CTGAAAAGCGCGGCGAGCCTGAACAGCCGCGTGGCGAGCATTAGCCTGCCGACCAGCTGCGC
GAGCGCGGGCACCCAGTGCCTGATTAGCGGCTGGGGCAACACCAAAAGCAGCGGCACCAGCT
ATCCGGATGTGCTGAAATGCCTGAAAGCGCCGATTCTGAGCGATAGCAGCTGCAAAAGCGCG
TATCCGGGCCAGATTACCAGCAACATGTTTTGCGCGGGCTATCTGGAAGGCGGCAAAGATAG
CTGCCAGGGCGATAGCGGCGGCCCGGTGGTGTGCAGCGGCAAACTGCAGGGCATTGTGAGC
TGGGGCAGCGGCTGCGCGCAGAAAAACAAACCGGGCGTGTATACCAAAGTGTGCAACTATGT
GAGCTGGATTAAACAGACCATTGCGAGCAACTAGAAGCTT
SEQ ID NO.: 2 (L-chain of BoNT/E)
PKINSFNYND PVNDRTILYI KPGGCQEFYK SFNIMKNIWI IPERNVIGTT
PQDFHPPTSL KNGDSSYYDP NYLQSDEEKD RFLKIVTKIF NRINNNLSGG
ILLEELSKAN PYLGNDNTPD NQFHIGDASA VEIKFSNGSQ DILLPNVIIM
GAEPDLFETN SSNISLRNNY MPSNHGFGSI AIVTFSPEYS FRFNDNSMNE
FIQDPALTLM HELIHSLHGL YGAKGITTKY TITQKQNPLI TNIRGTNIEE
FLTFGGTDLN IITSAQSNDI YTNLLADYKK IASKLSKVQV SNPLLNPYKD
VFEAKYGLDK DASGIYSVNI NKFNDIFKKL YSFTEFDLAT KFQVKCRQTY
IGQYKYFKLS NLLNDSIYNI SEGYNINNLK VNFRGQNANL NPRIITPITG
RGLVKKIIRF CKNIVSVKGI R
SEQ ID: 3 (H-chain of BoNT/E)

| | |
|---|---|
| 1 | KSICIEINNG ELFFVASENS YNDDNINTPK EIDDTVTSNN NYENDLDQVI |
| 51 | LNFNSESAPG LSDEKLNLTI QNDAYIPKYD SNGTSDIEQH DVNELNVFFY |
| 101 | LDAQKVPEGE NNVNLISSID TALLEQPKIY TFFSSEFINN VNKPVQAALF |
| 151 | VSWIQQVLVD FTTEANQKST VDKIADISIV VPYIGLALNI GNEAQKGNFK |
| 201 | DALELLGAGI LLEFEPELLI PTILVFTIKS FLGSSDNKNK VIKAINNALK |
| 251 | ERDEKWKEVY SFIVSNWMTK INTQFNKRKE QMYQALQNQV NAIKTIIESK |
| 301 | YNSYTLEEKN ELINKYDIKQ IENELNQKVS IAMNNIDRFL TESSISYLMK |
| 351 | LINEVKINKL REYDENVKTY LLNYIIQHGS ILGESQQELN SMVTDTLNNS |
| 401 | IPFKLSSYTD DKILISYFNK FFKRIKSSSV LNMRYKNDKY VDTSGYDSNI |
| 451 | NINGDVYKYP TNKNQFGIYN DKLSEVNISQ NDYIIYDNKY KNFSISFWVR |
| 501 | IPNYDNKIVN VNNEYTIINC MRDNNSGWKV SLNHNEIIWT LQDNAGINQK |
| 551 | LAFNYGNANG ISDYINKWIF VTITNDRLGD SKLYINGNLI DQKSILNLGN |
| 601 | IHVSDNILFK IVNCSYTRYI GIRYFNIFDK ELDETEIQTL YSNEPNTNIL |
| 651 | KDFWGNYLLY DKEYYLLNVL KPNNFIDRRK DSTLSINNIR STILLANRLY |
| 701 | SGIKVKIQRV NNSSINDNLV RKNDQVYINF VASKTHLFPL YADTATTNKE |
| 751 | KTIKISSSGN RFNQVVVMNS VGNNCTMNFK NNNGNNIGLL GFKADTVVAS |
| 801 | TWYYTHMRDH TNSNGCFWNF ISEEHGWQEK |

SEQ ID: 4 (5x c-terminal residues of TAP)
Asp-Asp-Asp-Asp-Lys
SEQ ID: 5 (6x c-terminal residues of TAP)
Val-Asp-Asp-Asp-Asp-Lys

1. A method for producing β-trypsin, the method comprising:
(a) renaturing denatured trypsinogen to produce renatured trypsinogen;
(b) purifying the renatured trypsinogen by anion exchange chromatography, thereby providing purified renatured trypsinogen; and
(c) incubating the purified renatured trypsinogen under conditions that promote its proteolytic activity, thereby leading to its cleavage into β-trypsin;
wherein:
steps (a) and (b) are carried out under conditions that do not promote the proteolytic activity of trypsinogen;
the method does not comprise the addition of a further protease for cleaving trypsinogen into β-trypsin;
the renaturing in step (a) is carried out in a buffer comprising L-arginine;
at least step (c) is carried out in a buffer that does not comprise L-arginine; and
prior to step (c), when present in a buffer that does not comprise L-arginine, the trypsinogen is not subjected to a temperature of >8° C. for more than 38 hours.

2. The method of claim 1, wherein the following steps are carried out prior to step (a):
(i) culturing a prokaryotic host cell comprising a nucleotide sequence encoding trypsinogen, wherein the nucleotide sequence is operably linked to an inducible promoter;
(ii) inducing expression of the trypsinogen by the host cell, thereby forming one or more insoluble inclusion bodies comprising the trypsinogen;
(iii) isolating the one or more insoluble inclusion bodies from the host cell;
(iv) solubilising the one or more insoluble inclusion bodies, thereby producing denatured trypsinogen; and
(v) purifying the denatured trypsinogen by cation exchange chromatography, thereby providing purified denatured trypsinogen.

3. A method for producing β-trypsin, the method comprising:
(a) culturing a prokaryotic host cell comprising a nucleotide sequence encoding trypsinogen, wherein the nucleotide sequence is operably linked to an inducible promoter;
(b) inducing expression of the trypsinogen by the host cell, thereby forming one or more insoluble inclusion bodies comprising the trypsinogen;
(c) isolating the one or more insoluble inclusion bodies from the host cell;
(d) solubilising the one or more insoluble inclusion bodies, thereby producing denatured trypsinogen;
(e) purifying the denatured trypsinogen by cation exchange chromatography, thereby providing purified denatured trypsinogen;
(f) renaturing the purified denatured trypsinogen to produce renatured trypsinogen;
(g) purifying the renatured trypsinogen by anion exchange chromatography, thereby providing purified renatured trypsinogen;
(h) incubating the purified renatured trypsinogen under conditions that promote its proteolytic activity, thereby leading to its cleavage into β-trypsin; and
(i) isolating the β-trypsin by affinity chromatography;
wherein:
steps (f) and (g) are carried out under conditions that do not promote the proteolytic activity of the trypsinogen; and
the method does not comprise the addition of a further protease for cleaving trypsinogen into β-trypsin.

4. The method of claim 1, further comprising subjecting the renatured trypsinogen to a volume reduction step.

5. The method of claim 1, further comprising subjecting the renatured trypsinogen to a filtration step that removes molecules having a size of less than 20 kDa.

6. (canceled)

7. The method of claim 1, wherein the step of incubating the purified renatured trypsinogen under conditions that promote its proteolytic activity is carried out up to 60 minutes after the purification of the renatured trypsinogen.

8. The method of claim 1, wherein the proteolytic activity of the purified renatured trypsinogen is promoted by adding calcium.

9. (canceled)

10. The method of claim 3, wherein the step of renaturing denatured trypsinogen is carried out in a buffer that comprises L-arginine.

11. The method of claim 3, wherein the step of incubating the purified renatured trypsinogen under conditions that promote its proteolytic activity is carried out in a buffer that does not comprise L-arginine.

12. The method of claim 3, wherein prior to the step of incubating the purified renatured trypsinogen under conditions that promote its proteolytic activity, the trypsinogen, when present in a buffer that does not comprise L-arginine, is not subjected to a temperature of 15-30° C. for more than 2 hours.

13. The method of claim 1, wherein in the step of renaturing the purified denatured trypsinogen, L-arginine is present at a concentration of 0.6 M to <1 M.

14-15. (canceled)

16. The method of claim 1, further comprising isolating the β-trypsin.

17. (canceled)

18. The method of claim 1, wherein steps (a) and (b) are carried out in the absence of calcium.

19. The method of claim 3, wherein steps (f) and (g) are carried out in the absence of calcium.

20. (canceled)

21. The method of claim 1, wherein the trypsinogen and β-trypsin are bovine trypsinogen and bovine β-trypsin, respectively.

22. The method according of claim 1, wherein the prokaryotic host cells are Escherichia coli host cells.

23-27. (canceled)

28. The method of claim 1, wherein, in the step of incubating the purified renatured trypsinogen under conditions that promote its proteolytic activity, calcium is added to a final concentration of at least 10 mM.

29-31. (canceled)

32. The method of claim 1, further comprising contacting a single-chain clostridial neurotoxin with the β-trypsin, wherein the single-chain clostridial neurotoxin comprises an activation loop and the β-trypsin hydrolyses a peptide bond of the activation loop, resulting in the formation of a di-chain clostridial neurotoxin.

33. A β-trypsin composition produced using the method of claim 32, wherein at least 80% of the total polypeptides in the composition are β-trypsin and wherein the composition has an activity level of at least 3000 USP units/mg of total polypeptides.

34. A method for producing a di-chain clostridial neurotoxin, the method comprising contacting a single-chain clostridial neurotoxin having an activation loop with the β-trypsin composition of claim 33; wherein the β-trypsin hydrolyses a peptide bond of the activation loop of the single-chain clostridial neurotoxin, thereby resulting in the formation of the di-chain clostridial neurotoxin.

35. (canceled)


SEQUENCE LISTING (5 sequences)

SEQ ID NO: 1 - DNA, 721 bp, Bos taurus
catatgtagaaggagatataccatggtggatgatgatgataaaattgtgggcggctatacctgcggcgcgaacaccgtgccgtatcaggtgagcctgaacagcggctatcatttttgcggcggcagcctgattaacagccagtgggtggtgagcgcggcgcattgctataaaagcggcattcaggtgcgcctgggcgaagataacattaacgtggtggaaggcaacgaacagtttattagcgcgagcaaaagcattgtgcatccgagctataacagcaacaccctgaacaacgatattatgctgattaaactgaaaagcgcggcgagcctgaacagccgcgtggcgagcattagcctgccgaccagctgcgcgagcgcgggcacccagtgcctgattagcggctggggcaacaccaaaagcagcggcaccagctatccggatgtgctgaaatgcctgaaagcgccgattctgagcgatagcagctgcaaaagcgcgtatccgggccagattaccagcaacatgttttgcgcgggctatctggaaggcggcaaagatagctgccagggcgatagcggcggcccggtggtgtgcagcggcaaactgcagggcattgtgagctggggcagcggctgcgcgcagaaaaacaaaccgggcgtgtataccaaagtgtgcaactatgtgagctggattaaacagaccattgcgagcaactagaagctt

SEQ ID NO: 2 - protein, 421 aa, Clostridium botulinum
PKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 3 - protein, 830 aa, Clostridium botulinum
KSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 4 - protein, 5 aa, Artificial Sequence
  5x c-terminal residues of TAP
DDDDK

SEQ ID NO: 5 - protein, 6 aa, Artificial Sequence
  6x c-terminal residues of TAP
VDDDDK